(19) United States
(12) Patent Application Publication
(10) Pub. No.: US 2013/0287854 A1
(43) Pub. Date: Oct. 31, 2013

(54) COMPOSITIONS AND USES

(71) Applicants: Frazer Giles Morgan, Chippenham (GB); Mark Jonathan Main, Chippenham (GB)

(72) Inventors: Frazer Giles Morgan, Chippenham (GB); Mark Jonathan Main, Chippenham (GB)

(73) Assignee: VECTURA LIMITED, Chippenham, Wiltshire (GB)

(21) Appl. No.: 13/885,531

(22) PCT Filed: Nov. 15, 2011
(86) PCT No.: PCT/GB2011/052222

§ 371 Date: Jun. 26, 2013

(30) Foreign Application Priority Data
Nov. 15, 2010 (GB) 1019291.2
Feb. 4, 2011 (GB) 1101924.7
May 5, 2011 (GB) 1107454.9

(51) Int. Cl. A61K 9/00; A61K 31/277; A61K 45/06; A61K 31/473; A61K 31/198

(52) U.S. Cl. CPC A61K 9/0075; A61K 31/473; A61K 31/198; A61K 45/06; A61K 31/277
USPC 424/489; 514/284; 514/250; 514/220

(57) ABSTRACT

According to the invention there is provided a method of treating and/or preventing the symptoms of Parkinson's disease comprising delivering apomorphine, optionally in combination with levodopa and/or a dopamine agonist that is not apomorphine, wherein apomorphine is administered by inhalation.

The present invention relates to compositions for providing improved treatment of diseases and disorders of the central nervous system, including Parkinson's disease.

### BACKGROUND OF THE INVENTION

Parkinson's disease (PD) was first described in England in 1817 by Dr James Parkinson. The disease affects approximately 2 of every 1,000 people and most often develops in those over 50 years of age, affecting both men and women. It is one of the most common neurological disorders of the elderly, and occasionally occurs in younger adults. In some cases, Parkinson's disease occurs within families, especially when it affects young people. Most of the cases that occur at an older age have no known cause.

The specific symptoms that an individual experiences vary, but may include tremor of the hands, arms, legs, jaw and face; rigidity or stiffness of the limbs and trunk; bradykinesia or slowness of movement; postural instability or impaired balance and coordination as well as severe depression. Untreated, Parkinson's disease progresses to total disability, often accompanied by general deterioration of all brain functions, and may lead to an early death.

The symptoms of Parkinson's disease result from the loss of dopamine-secreting (dopaminergic) cells in the substantia nigra of the upper part of the brainstem. The exact reason for the wasting of these cells is unknown, although both genetic and environmental factors are known to be important.

There is no known cure for Parkinson's disease. The goal of treatment is to control symptoms, and medications aim to do this primarily by increasing the levels of dopamine in the brain. The most widely used treatment is L-dopa (also known as levodopa) in various forms. However, this treatment has a number of drawbacks, the most significant being that, due to feedback inhibition, L-dopa results in a reduction in the endogenous formation of L-dopa (and hence dopamine), and so eventually becomes counterproductive. Over time, patients start to develop motor fluctuations, which oscillate between “off” episodes, a state of decreased mobility, and “on” episodes, or episodes when the medication is working and symptoms are controlled. It is estimated that over 50% of Parkinson's patients will experience motor fluctuations within 4-6 years of onset, increasing by 10 percent per year after that.

The average Parkinson's disease patient experiences 2-3 hours of “off-episodes” each day. These include handwriting problems, overall slowness, loss of olfaction, loss of energy, stiffness of muscles, walking problems, sleep disturbances, balance difficulties, challenges getting up from a chair, and many other symptoms not related to motor functions, such as sensory symptoms (e.g. pain, fatigue, and motor restlessness); autonomic symptoms (e.g. urinary incontinence and profuse sweats); and psychiatric disorders (e.g. depression, anxiety and psychosis).

One therapeutic approach involves the administration of apomorphine, which is a morphine derivative and dopaminergic agonist. First mooted as a treatment for Parkinson's disease as early as 1951, the first clinical use of apomorphine was first reported in 1970 by Cotzias et al (New England Journal of Medicine 282(1): 31-3), although its emetic properties, short half-life and significant first-pass metabolism in the gastrointestinal (GI) tract made oral use impractical.

The use of apomorphine to treat Parkinson's disease is effective because of the drug's strong dopaminergic action. However, sublingually administered apomorphine is associated with an onset period of about 30 to 45 minutes during which the patient suffers unnecessarily. Now, a more common route of administration is by subcutaneous injection. Data from Apokyn® marketing literature has claimed that 90% of patients experienced improved movement within 20 minutes post dose.

Whilst apomorphine can be used in combination with L-dopa, the usual intention in the later stages of the disease is to wean patients off L-dopa, as by this stage they will probably be experiencing significant discomfort from off-episodes.

Apomorphine has a low incidence of neuropsychiatric problems, and it has thus been used in patients with severe neuropsychiatric complications due to oral anti-Parkinsonian drugs. Injections of apomorphine may help specific symptoms such as off-period pain, belching, screaming, constipation, nocturia, dystonias, erectile impotence, and post-surgical state in selected patients who may not otherwise be candidates for apomorphine.

In the US prescribing instructions for subcutaneous administration, the usual dose of apomorphine is 2 mg (provided in a volume of 0.2 ml) per delivery, and it is not recommended to exceed 6 mg in a single off-period because the risk of sensitisation to apomorphine does not outweigh the benefit of the larger doses. The British National Formulary (BNF) recommends that the usual range (after initiation) of a subcutaneous injection is 3 to 30 mg per day to be administered in divided doses. Subcutaneous infusion may be preferable in those patients requiring division of injections into more than 10 doses daily. The maximum single dose is 10 mg, with a total daily dose by either subcutaneous route (or combined routes) that is not to exceed 100 mg.

The recommended continuous subcutaneous infusion dose is initially 1 mg/hour daily and is generally increased according to response (not more often than every 4 hours) in maximum steps of 500 μg/hour, to a usual rate of 1 to 4 mg/hour (14 to 60 μg/kg/hour). The infusion site is to be changed every 12 hours and infusion is to be given during waking hours only; 24-hour infusions are not advised unless the patient experiences severe night-time symptoms. Intermittent bolus boosts may also be needed.

However, frequent injection of low doses of apomorphine are often inadequate in controlling the disease symptoms, and in addition to the pain caused by repeated injection, these repeated injections inconvenience the patient, often resulting in non-compliance.

Apomorphine can be administered via subcutaneous infusion using a small pump which is carried by the patient. A low dose is automatically administered throughout the day, reducing the fluctuations of motor symptoms by providing a steady dose of dopaminergic stimulation. However, an additional person (often a spouse or partner) must be responsible for maintenance of the pump, placing a burden on this caregiver.

Adverse effects (AEs) commonly observed with apomorphine administration include nausea and vomiting, and hypotension. Yawning, dyskinesia and somnolence may also be reported at similar if not higher incidence levels. In light of these AEs, the BNF reports that patients are often given anti-emetic prophylaxis three days prior to the initiation of apomorphine therapy and it is recommended that this continue for eight weeks after the apomorphine treatment has finished. Furthermore drowsiness (including sudden onset of sleep), confusion, hallucinations, injection-site reactions (including nodule formation and ulceration), less commonly postural hypotension, breathing difficulties, dyskinesia during “on” episodes, haemolytic anaemia with levodopa, rarely eosinophilia, pathological gambling, increased libido and hypersexuality are also reported.

Anti-emetic therapies that may be used include domperidone or trimethobenzamide (trade name Tigan).

The term “parkinsonism” refers to any condition that involves a combination of the types of changes in movement seen in Parkinson's disease and often has a specific cause, such as the use of certain drugs or frequent exposure to toxic chemicals. Generally, the symptoms of parkinsonism may be treated with the same therapeutic approaches that are applied to Parkinson's disease.

A dry powder formulation suitable for intranasal delivery of apomorphine is the focus of European Patent No. 0 689 438. The powder formulation comprises particles of apomorphine having a diameter in the range of 50-100 μm in order to avoid accidental pulmonary deposition. Published studies by Genus, formerly Britannia Pharmaceuticals Ltd, examined the use of nasally administered apomorphine compositions of this kind and have indicated that the onset of pharmaceutical effects is delayed, and the efficacy of these medicaments is reduced in comparison to subcutaneously delivered apomorphine in terms of the percentage decrease in off-period time. Furthermore, some nasal irritation was reported.

Nasal apomorphine formulations have been evaluated by Nastech Inc. for the treatment of Erectile Dysfunction (ED) and Female Sexual Dysfunction (FSD). Although this route of administration presents advantages over the conventional sublingual route of administering apomorphine for treating this condition, intranasal administration does have a number of drawbacks.

The nasal cavity presents a significantly reduced available surface area compared to the lung (1.8 m² versus 200 m²). The nasal cavity is also subjected to natural clearance, which typically occurs every 15-20 minutes, where ciliated cells drive mucus and debris towards the back of the nasopharynx. This action results in a proportion of the apomorphine which is administered to the nose being swallowed, whereupon it is subjected to first-pass metabolism. In contrast, clearance mechanisms in the lung have minimal opportunity to influence absorption as apomorphine rapidly reaches the systemic circulation via transfer across the alveolar membrane.

Challenges to the nasal mucosa, such as congestion or a “bloody” nose will also have a negative impact upon drug absorption following nasal administration. Furthermore, the nasal passage shape and dimension influence drug absorption. Not only are the passages different between patients but there is also a change in shape and dimensions within a patient at different times during the day. Consequently, nasal delivery devices must overcome this significant challenge to ensure reproducible and targeted drug delivery. To ensure delivery to the target site nasal devices typically employ a “forceful” spray which can result in an undesirable sensation. Conversely, inhalers, including dry powder inhalers such as the Vectura's active inhaler device Aspirair® or their passive device Gyrohaler®, produce a patient-friendly drug “cloud” with minimal oral and throat deposition. Additional dry powder inhalers that may be mentioned include those described in WO 2010/086285.

Furthermore, extensive literature describes local apomorphine-attributed irritation following intranasal administration with a number of patients reporting episodes of severe or disabling nasal complications including irritation, crusting, blockage, bleeding, burning immediately after dosing and vestibulitis leading to premature discontinuation of study treatment.

Nevertheless, the apomorphine nasal powder developed by Genus, formerly Britannia Pharmaceuticals Ltd, is said to offer a rapid onset that is comparable to subcutaneous injection and much faster than oral dosing, as well as bioavailability that is also comparable to the subcutaneous route of administration.

U.S. Pat. No. 6,193,954 (Abbott Laboratories) relates to formulations for pulmonary delivery of dopamine agonists. The dopamine agonist is in the form of a microparticle or powder and is to be delivered to the lung dispersed in a liquid vehicle.

U.S. Pat. No. 6,514,482 (Advanced Inhalation Research, Inc.) claims a method of providing “rescue therapy” in the treatment of Parkinson's disease in which particles of apomorphine are delivered to the pulmonary system. Rescue therapy normally refers to non-surgical medical treatment in life-threatening situations. However, despite the unpleasantness of Parkinson's disease, the symptoms are not life threatening and this patent would therefore appear to relate to “rescue” from off-episode symptoms. As used within U.S. Pat. No. 6,514,482, “rescue therapy” means on-demand, rapid delivery of a drug to a patient to help reduce or control disease symptoms.

In the prior art, the dopamine agonist compositions and the methods of treating Parkinson's disease involve administering fixed doses of apomorphine at the onset of off-episode symptoms. This does not provide the optimal treatment. It would be highly beneficial to be able to readily determine the appropriate dose of apomorphine to suit the specific needs of an individual patient, or if the patient is using apomorphine in combination with other agents that treat the symptoms of Parkinson's disease. This would ensure that the minimum necessary dose is administered. Such a self-titrating system should be flexible, to enable the dose to be tailored to the patient without the need for different strength presentations. The system should also allow the self-titrating to be on-going, with the patient able to constantly change the dose of apomorphine to meet his or her symptoms and needs. This is desirable for a number of reasons, not least in order to minimise the adverse side effects associated with the treatment (including emesis, reduced sleep and dyskinesia) and to reduce the risk of apomorphine sensitisation.

It is a further aim to reduce “off-episodes” experienced by the patient as much as possible and, if possible, to avoid such off-episodes altogether. It is desirable to achieve this without the need to administer excessively large doses of apomorphine, especially in terms of the daily dose administered to the patient over a 24-hour period and optionally when apomorphine is used in combination with other agents that treat the symptoms of Parkinson's disease. A particularly advantageous effect of an inhaled apomorphine composition and treatment regimen would be to reduce the time a patient spends in an “off episode” each day in comparison with other apomorphine treatment regimens (such as administration of apomorphine by subcutaneous injection).

It is also clearly desirable to provide a composition or treatment regimen which the patient is able to self-administer, reducing the burden on the care-giver. A safe, convenient, non-invasive and pain-free route of administration is clearly preferable to constant and frequent injections or a permanent infusion pump. A medication which alleviates this dependency while allowing ease of delivery for frequent administration of apomorphine would clearly be an advantage.

A formulation that is capable of maintaining an extended duration of response would provide the patient with a window in which they could self administer the next dose, thereby negate the need for caregiver assistance.

A method of administration which reduces the emetic effects of apomorphine would be advantageous.

A method that provides a superior safety profile and a reduced incidence of typical AEs, particularly sleep deprivation and/or dyskinesia in patients with Parkinson's disease would be advantageous.

A dosing schedule that minimises the total daily dose of apomorphine, which is the dose administered over a period of 24 hours, while maximising therapeutic effectiveness in a patient would be of significant benefit. For example, by minimising the total amount of time spent in off-episodes.

Nasal administration of apomorphine results in a Tₘₐₓ of approximately 15 minutes. Pulmonary administration results in a Tₘₐₓ as rapid as 1 minute in some patients. This is thought to be equivalent if not faster to the Tₘₐₓ observed following subcutaneous (sc) administration. Pulmonary administration has greater bioavailability than nasal administration. This, in turn, means that nasal doses need to be increased in order to compensate for the lower bioavailability.

In the information sheet for Apokyn® dated April 2004, it is stated that apomorphine hydrochloride is a lipophilic compound that is rapidly absorbed (time to peak concentration ranges from 10 to 60 minutes) following subcutaneous administration into the abdominal wall. After subcutaneous administration, apomorphine appears to have bioavailability equal to that of an intravenous administration. Apomorphine exhibits linear pharmacokinetics over a dose range of 2 to 8 mg following a single subcutaneous injection of apomorphine into the abdominal wall in patients with idiopathic Parkinson's disease.

Based upon the assertion that the bioavailability of subcutaneously administered apomorphine is equal to that of intravenously administered apomorphine, it is surprising that the bioavailability of apomorphine administered by pulmonary inhalation is comparable, if not greater than the bioavailability following subcutaneous injection.

There is still a need for improved Parkinson's disease therapies.

### SUMMARY OF THE INVENTION

In a first aspect of the present invention, there is provided a method of treating and/or preventing the symptoms of Parkinson's disease (PD) comprising delivering apomorphine in combination with levodopa and/or a dopamine agonist that is not apomorphine, wherein apomorphine is administered by inhalation.

Thus according to further aspects of the present invention, there is provided:

(I) a combination of apomorphine with levodopa and/or a dopamine agonist that is not apomorphine for use in treating and/or preventing the symptoms of Parkinson's disease, wherein apomorphine is administered by inhalation;
(II) a kit comprising apomorphine, levodopa and/or a dopamine agonist that is not apomorphine for use in treating and/or preventing the symptoms of Parkinson's disease, wherein apomorphine is administered by inhalation; and
(III) a use comprising an effective amount of apomorphine in combination with an effective amount of levodopa and/or a dopamine agonist that is not apomorphine in the preparation of a medicament for treating and/or preventing the symptoms of Parkinson's disease, wherein apomorphine is administered by inhalation.

In a further aspect of the present invention, there is provided a method of treating and/or preventing the symptoms of Parkinson's disease comprising delivering apomorphine, optionally in combination with levodopa and/or a dopamine agonist that is not apomorphine, wherein apomorphine is administered by inhalation and the maximum daily dose of apomorphine is less than 30 mg (e.g. 27 mg, such as 24.5 mg or, particularly, 22.5 mg).

Thus according to further aspects of the present invention, there is provided:

(I) apomorphine, optionally with levodopa and/or a dopamine agonist that is not apomorphine, for use in treating and/or preventing the symptoms of Parkinson's disease, wherein apomorphine is administered by inhalation and the maximum daily dose of apomorphine is less than 30 mg;
(II) a kit comprising apomorphine, optionally levodopa and/or a dopamine agonist that is not apomorphine, for use in treating and/or preventing the symptoms of Parkinson's disease, wherein apomorphine is administered by inhalation and the maximum daily dose of apomorphine is less than 30 mg; and
(III) a use comprising an effective amount of apomorphine, optionally in combination with an effective amount of levodopa and/or a dopamine agonist that is not apomorphine, in the preparation of a medicament for treating and/or preventing the symptoms of Parkinson's disease, wherein apomorphine is administered by inhalation and the maximum daily dose of apomorphine is less than 30 mg.

The dose is suitably a fine particle dose, measured as described herein, but may be a nominal dose.

In yet a further aspect of the present invention, there is provided a method of treating and/or preventing the symptoms of Parkinson's disease comprising delivering apomorphine, optionally in combination with levodopa and/or a dopamine agonist that is not apomorphine, wherein apomorphine is administered by inhalation and the apomorphine is delivered in a fine particle dose of between 0.5 to 4.5 mg (e.g. 0.5 to 3.5 mg).

Thus according to further aspects of the present invention, there is provided:

(I) apomorphine, optionally in combination with levodopa and/or a dopamine agonist that is not apomorphine, for use in treating and/or preventing the symptoms of Parkinson's disease, wherein apomorphine is administered by inhalation and the apomorphine is delivered in a fine particle dose of between 0.5 to 4.5 mg;
(II) a kit comprising apomorphine, optionally levodopa and/or a dopamine agonist that is not apomorphine, for use in treating and/or preventing the symptoms of Parkinson's disease, wherein apomorphine is administered by inhalation and the apomorphine is delivered in a fine particle dose of between 0.5 to 4.5 mg; and
(III) a use comprising an effective amount of apomorphine, optionally in combination with an effective amount of levodopa and/or a dopamine agonist that is not apomorphine, in the preparation of a medicament for treating and/or preventing the symptoms of Parkinson's disease, wherein apomorphine is administered by inhalation and the apomorphine is delivered in a fine particle dose of between 0.5 to 4.5 mg.

In yet a further aspect of the present invention, there is provided a method of treating and/or preventing the symptoms of Parkinson's disease comprising delivering an inhaled apomorphine formulation optionally in combination with levodopa and/or a dopamine agonist that is not apomorphine, wherein:
(a) the apomorphine formulation is able to produce an apomorphine Cₘₐₓ within 1 to 10 minutes (e.g. 1 to 5 minutes, such as 1 to 3 minutes) of inhalation; and
(b) the concentration of apomorphine in the blood decreases to no more than 80% of Cₘₐₓ within 4 minutes of Cₘₐₓ being achieved.

Thus according to further aspects of the present invention, there is provided:

(I) apomorphine, optionally in combination with levodopa and/or a dopamine agonist that is not apomorphine, for use in treating and/or preventing the symptoms of Parkinson's disease, wherein:
(a) the apomorphine is administered by inhalation and the apomorphine formulation is able to produce an apomorphine Cₘₐₓ within 1 to 10 minutes (e.g. 1 to 5 minutes, such as 1 to 3 minutes) of inhalation; and
(b) the concentration of apomorphine in the blood decreases to no more than 80% of Cₘₐₓ within 4 minutes of Cₘₐₓ being achieved;
   (II) a kit comprising apomorphine, optionally levodopa and/or a dopamine agonist that is not apomorphine, for use in treating and/or preventing the symptoms of Parkinson's disease, wherein:
(a) the apomorphine is administered by inhalation and the apomorphine formulation is able to produce an apomorphine Cₘₐₓ within 1 to 10 minutes (e.g. 1 to 5 minutes, such as 1 to 3 minutes) of inhalation; and
(b) the concentration of apomorphine in the blood decreases to no more than 80% of Cₘₐₓ within 4 minutes of Cₘₐₓ being achieved; and
   (III) a use comprising an effective amount of apomorphine, optionally in combination with an effective amount of levodopa and/or a dopamine agonist that is not apomorphine, in the preparation of a medicament for treating and/or preventing the symptoms of Parkinson's disease, wherein:
(a) the apomorphine is administered by inhalation and the apomorphine formulation is able to produce an apomorphine Cₘₐₓ within 1 to 10 minutes (e.g. 1 to 5 minutes, such as 1 to 3 minutes) of inhalation; and
(b) the concentration of apomorphine in the blood decreases to no more than 80% of Cₘₐₓ within 4 minutes of Cₘₐₓ being achieved.

In still yet a further aspect of the present invention, there is provided a method of reducing sleep loss in patients with Parkinson's disease comprising delivering apomorphine, optionally in combination with levodopa and/or a dopamine agonist that is not apomorphine, wherein apomorphine is administered by inhalation.

Thus according to further aspects of the present invention, there is provided:

(I) apomorphine, optionally in combination with levodopa and/or a dopamine agonist that is not apomorphine, for use in reducing sleep loss in patients with Parkinson's disease, wherein apomorphine is administered by inhalation;
(II) a kit comprising apomorphine, optionally levodopa and/or a dopamine agonist that is not apomorphine, for use in reducing sleep loss in patients with Parkinson's disease, wherein apomorphine is administered by inhalation; and
(III) a use comprising an effective amount of apomorphine, optionally in combination with an effective amount of levodopa and/or a dopamine agonist that is not apomorphine, in the preparation of a medicament for reducing sleep loss in patients with Parkinson's disease, wherein apomorphine is administered by inhalation.

In a still further aspect of the present invention, there is provided a method of reducing off-episodes in patients with Parkinson's disease comprising delivering apomorphine by inhalation, optionally in combination with levodopa and/or a dopamine agonist that is not apomorphine.

Thus according to further aspects of the present invention, there is provided:

(I) apomorphine, optionally in combination with levodopa and/or a dopamine agonist that is not apomorphine, for use in reducing off-episodes in patients with Parkinson's disease, wherein the apomorphine is delivered by inhalation;
(II) a kit comprising apomorphine for inhalation, optionally levodopa and/or a dopamine agonist that is not apomorphine, for reducing off-episodes in patients with Parkinson's disease, wherein the apomorphine is delivered by inhalation; and
(III) a use comprising an effective amount of apomorphine for inhalation, optionally in combination with an effective amount of levodopa and/or a dopamine agonist that is not apomorphine, in the preparation of a medicament reducing off-episodes in patients with Parkinson's disease wherein the apomorphine is delivered by inhalation.

In a still further aspect of the present invention, there is provided a method of reducing dyskinesia in patients with Parkinson's disease comprising delivering apomorphine by inhalation, optionally in combination with levodopa and/or a dopamine agonist that is not apomorphine.

Thus according to further aspects of the present invention, there is provided:

(I) apomorphine, optionally in combination with levodopa and/or a dopamine agonist that is not apomorphine, for use in reducing dyskinesia in patients with Parkinson's disease, wherein the apomorphine is delivered by inhalation;
(II) a kit comprising apomorphine for inhalation, optionally levodopa and/or a dopamine agonist that is not apomorphine, for reducing dyskinesia in patients with Parkinson's disease, wherein the apomorphine is delivered by inhalation; and
(III) a use comprising an effective amount of apomorphine for inhalation, optionally in combination with an effective amount of levodopa and/or a dopamine agonist that is not apomorphine, in the preparation of a medicament reducing dyskinesia in patients with Parkinson's disease wherein the apomorphine is delivered by inhalation.

In yet a further aspect of the present invention, there is provided a method of treating and/or preventing the symptoms of a disease associated with a dopamine agonist deficiency comprising delivering an inhaled dopamine agonist formulation optionally in combination with levodopa and/or a different dopamine agonist, wherein:
(c) the dopamine agonist formulation is able to produce an apomorphine Cₘₐₓ within 1 to 10 minutes (e.g. 1 to 5 minutes, such as 1 to 3 minutes) of inhalation; and
(d) the concentration of the dopamine agonist in the blood decreases to no more than 80% of Cₘₐₓ within 4 minutes of Cₘₐₓ being achieved.

Thus according to further aspects of the present invention, there is provided:

(I) a dopamine agonist, optionally in combination with levodopa and/or a different dopamine agonist, for use in treating and/or preventing the symptoms of Parkinson's disease, wherein:
(a) the dopamine agonist is administered by inhalation and the apomorphine formulation is able to produce an apomorphine Cₘₐₓ within 1 to 10 minutes (e.g. 1 to 5 minutes, such as 1 to 3 minutes) of inhalation; and
(b) the concentration of the dopamine agonist in the blood decreases to no more than 80% of Cₘₐₓ within 4 minutes of Cₘₐₓ being achieved;
   (II) a kit comprising a dopamine agonist, optionally levodopa and/or a different dopamine agonist, for use in treating and/or preventing the symptoms of Parkinson's disease, wherein:
(a) the dopamine agonist is administered by inhalation and the apomorphine formulation is able to produce an apomorphine Cₘₐₓ within 1 to 10 minutes (e.g. 1 to 5 minutes, such as 1 to 3 minutes) of inhalation; and
(b) the concentration of the dopamine agonist in the blood decreases to no more than 80% of Cₘₐₓ within 4 minutes of Cₘₐₓ being achieved; and
   (III) a use comprising an effective amount of a dopamine agonist, optionally in combination with an effective amount of levodopa and/or a different dopamine agonist, in the preparation of a medicament for treating and/or preventing the symptoms of Parkinson's disease, wherein:
(a) the dopamine agonist is administered by inhalation and the apomorphine formulation is able to produce an apomorphine Cₘₐₓ within 1 to 10 minutes (e.g. 1 to 5 minutes, such as 1 to 3 minutes) of inhalation; and
(b) the concentration of the dopamine agonist in the blood decreases to no more than 80% of Cₘₐₓ within 4 minutes of Cₘₐₓ being achieved.

In an embodiment of the immediately preceding aspect, the disease is selected from one or more of Parkinson's disease, restless legs syndrome or cancer in the form of a pituitary tumour (e.g. Parkinson's disease).

In a yet still further aspect of the present invention, there is provided an inhaler device comprising an apomorphine composition as claimed in any one of the preceding claims (e.g. wherein the device is a dry powder inhaler, a pressurized metered dose inhaler or a nebuliser).

In a further aspect of the invention the Parkinsons patients to be treated in the present invention are patients have been diagnosed with Parkinson's disease for at least 5 years, and in one aspect for over 10 years.

### BRIEF DESCRIPTION OF THE DRAWINGS

FIG. 1 shows a table illustrating the demographic characteristics of active treatment groups and placebo groups from three independent phase II clinical studies. The VR040/2/003 and VR040/2/008 studies have been undertaken by Vectura Limited. APO202 is trial data from a study published in Arch Neurol 2001.

FIG. 2 is a table comparing active and placebo in-clinic UPDRS III changes from three independent phase II clinical studies (VR040/2/003, VR040/2/008 and APO202). The analysis utilises the Intent-to-Treat (ITT) patient populations.

FIG. 3 depicts the UPDRS III in-clinic changes in graphical format. The UPDRS III mean maximum change from the pre-dose is shown as a percentage. The analysis similarly utilises ITT patient populations.

FIG. 4 Illustrates the mean rapid and durable improvement in UPDRS III for the active treatment group to the placebo treatment group in the VR040/2/008 study over the period studied (in ITT patient populations).

FIG. 5 is a table comparing active and placebo in-clinic UPDRS III changes from three independent phase II clinical studies (VR040/2/003, VR040/2/008 and APO202). The analysis utilises Per-Protocol (PP) patient populations for VR040/2/003 and VR040/2/008 comparisons and ITT patient populations for the APO202 study.

FIG. 6 depicts the proportion of at home off episodes (ITT population) experienced by the active treatment group and the placebo treatment group in the VR040/2/008 study.

FIG. 7 is a table comparing the daily “off” episodes per day during the at-home dosing period of two independent phase II clinical studies (VR040/2/008 and APO202). ITT and PP patient populations were compared.

FIG. 8 depicts a comparison of the reduction in mean daily “off” episodes in hours compared with the baseline value in active treatment groups and placebo groups from VR040/2/008 and APO202 clinical studies. The analysis utilises ITT patient populations.

FIG. 9 shows a table illustrating the time to therapeutic benefit in the ITT patient population from three independent phase II clinical studies (VR040/2/003, VR040/2/008 and APO202). The analysis utilises ITT patient populations.

FIG. 10 is a table summarising the mean daily period of sleep experienced by active treatment groups and placebo groups in the VR040/2/008 and APO202 studies. The analysis utilises ITT patient populations.

FIG. 11 is a table summarising mean daily “on” episodes in which patients experience no dyskinesias, non-troublesome dyskinesias or troublesome dyskinesias. The VR040/2/008 and APO202 studies were compared, and analysis utilises ITT patient populations.

FIG. 12 represents the average time over a 24 hour day where a patient from the VR040/2/008 active treatment group is experiencing on-time, off-time, is either asleep or is experiencing dyskinesia.

FIG. 13 is a table summarising safety data, specifically the number and proportion of different patients with treatment-related adverse events (AEs) during the in-clinic and at-home VR040/2/008 study phases.

FIG. 14 shows the percentage of patients reporting AEs (in-clinic and at-home phases) in three independent clinical studies (VR040/2/008, APO202 and APO302).

FIG. 15 In clinic VR040/2/008 orthostatic challenge, change in the mean systolic blood pressure from pre-dose (ITT patient population).

FIG. 16 In clinic VR040/2/008 orthostatic challenge, change in the mean diastolic blood pressure from pre-dose (ITT patient population).

FIG. 17 In clinic VR040/2/008 orthostatic challenge, change in the mean pulse rate from pre-dose (ITT patient population).

FIG. 18 Summarises the number of patients with systolic blood pressure values of potential clinical concern (ITT patient population).

FIG. 19 Summarises the number of patients with diastolic blood pressure values of potential clinical concern (ITT patient population).

FIG. 20 Summarises the number of patients with pulse rate values of potential clinical concern (ITT patient population).

Summarises the number of patients with pulse rate values of potential clinical concern (ITT patient population).

FIG. 21 In clinic VR040/2/008 12 lead cardiac safety assessments (ITT patient population).

FIG. 22 Summarises the number of patients with ECG readings of potential clinical concern (ITT patient population).

FIG. 23 Illustrates the change in mean FEV1 (L) over the study period (ITT patient population).

FIG. 24 compares the mean daily “off” episodes (ITT patient population) in patients from two independent clinical studies (VR040/2/008 and a melevodopa/carbidopa study, published in Movement Disorders 2010).

FIG. 25 compares the active and placebo in-clinic UPDRS III changes from pulmonary (VR040/2/003 and VR040/2/008) and sublingual (S90049) administered apomorphine. The analysis utilises ITT patient populations.

FIG. 26 shows a typical pharmacokinetic profile for a patient treated with inhaled apomorphine in a recent clinical trial (VR040).

FIG. 27 is a schematic representation of the apomorphine pharmacokinetic profile observed in the VR040/2/003 study compared to subcutaneous administered apomorphine.

### DETAILED DESCRIPTION OF INVENTION

The present invention demonstrates the efficacy of using inhaled apomorphine in the treatment of patients with Parkinson's disease, demonstrating a benefit to patients when compared with injected apomorphine.

### DEFINITIONS

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients.

The term “effective amount” refers to an amount of a compound, which confers a therapeutic effect on the treated patient (e.g. sufficient to treat or prevent the disease).

The effect may be objective (i.e. measurable by some test or marker) or subjective (i.e. the subject gives an indication of or feels an effect).

When used herein, the term “Nominal Dose” (ND) is the amount of drug metered in the receptacle (also known as the Metered Dose). This is different to the amount of drug that is delivered to the patient which is referred to a Delivered Dose.

The fine particle fraction (FPF) is normally defined as the “fine particle dose” (FPD; the dose that is <5 μm) divided by the Emitted Dose (ED) which is the dose that leaves the device. The FPF is expressed as a percentage. Herein, the FPF of ED is referred to as FPF (ED) and is calculated as FPF (ED)=(FPD/ED)×100%.

FPD may be measured by a Multistage Liquid Impinger, United States Pharmacopoeia 26, Chapter 601, Apparatus 4 (2003), an Andersen Cascade Impactor or a New Generation Impactor.

When used herein, the term “fine particle fraction” (FPF) may also be defined as the FPD divided by the Metered Dose (MD) which is the dose in the blister or capsule, and expressed as a percentage. Herein, the FPF of MD is referred to as FPF (MD), and is calculated as FPF (MD)=(FPD/MD)×100%.

The term “ultrafine particle dose” (UFPD) is used herein to mean the total mass of active material delivered by a device which has a diameter of not more than 3 μm. The term “ultrafine particle fraction” is used herein to mean the percentage of the total amount of active material delivered by a device which has a diameter of not more than 3 μm. The term percent ultrafine particle dose (% UFPD) is used herein to mean the percentage of the total metered dose which is delivered with a diameter of not more than 3 μm (i.e., % UFPD=100×UFPD/total metered dose).

The terms “delivered dose” and “emitted dose” or “ED” are used interchangeably herein. These are measured as set out in the current European Pharmacopeia (EP) monograph for inhalation products.

“Actuation of an inhaler” refers to the process during which a dose of the powder is removed from its rest position in the inhaler. That step takes place after the powder has been loaded into the inhaler ready for use.

“Intent-to-Treat” (ITT) population refers to all patients who have been randomised and have received at least 1 dose of study treatment in the clinic.

“per-protocol” (PP) population refers to all patients in the ITT population who participate in the study with out major violation of the protocol.

The term “on” state with no dyskinesias refers to when a patient feels similar to how they felt before developing Parkinson's (in terms of normal motor function and the ability to do their regular activities).

The term “on” state with non-troublesome dyskinesias refers to when patient is in an “on” state with mild dyskinesias that are noticeable but do not interfere with their regular activities.

The term “on” state with non-troublesome dyskinesias refers to when a patient is in an “on” state with mild dyskinesias that are noticeable but do not interfere with regular activities.

The term “on” state with troublesome dyskinesias refers to when a patient is in an “on” state with dyskinesias that are severe enough to make regular activities somewhat difficult or very difficult.

The term “off” state refers to when a patient has stopped working so well, with the worsening of symptoms.

The term “Unified Parkinson's Disease Rating Scale” (UPDRS) is a rating tool to follow the longitudinal course of Parkinson's disease. It is made up of the 1) Mentation, Behavior, and Mood, 2) ADL and 3) Motor sections. These are evaluated by interview. Some sections require multiple grades assigned to each extremity. A total of 199 points are possible. 199 represents the worst (total) disability), 0—no disability.

### Possible Form of Active Agents

References herein (in any aspect or embodiment of the invention) to an active ingredient (such as apomorphine, levodopa, carbidopa, entacapone) includes references to such active ingredients per se, to tautomers of such compounds, as well as to pharmaceutically acceptable salts or solvates, or pharmaceutically functional derivatives of such active ingredients.

Pharmaceutically acceptable salts that may be mentioned include acid addition salts and base addition salts. Such salts may be formed by conventional means, for example by reaction of a free acid or a free base form of an active ingredient (e.g. apomorphine, levodopa, cardidopa etc) with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of an active ingredient (e.g. apomorphine, leveodopa, carbidopa etc) in the form of a salt with another counter-ion, for example using a suitable ion exchange resin.

Examples of pharmaceutically acceptable salts include acid addition salts derived from mineral acids and organic acids, and salts derived from metals such as sodium, magnesium, or particularly, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or particularly, potassium and calcium.

As mentioned above, the active agents discussed herein also includes any solvates of the active ingredients and their salts. Particular solvates that may be mentioned herein are solvates formed by the incorporation into the solid state structure (e.g. crystal structure) of the active agents described herein of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the active ingredient with a solvent or mixture of solvents containing the solvating solvent. Whether or not a solvate has been formed in any given instance can be determined by subjecting crystals of the active ingredient to analysis using well known and standard techniques such as thermogravimetric analysis (TGE), differential scanning calorimetry (DSC) and X-ray crystallography.

The solvates can be stoichiometric or non-stoichiometric solvates. Particular solvates that may be mentioned herein are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

“Pharmaceutically functional derivatives” of the active ingredients as defined herein includes ester derivatives and/or derivatives that have, or provide for, the same biological function and/or activity as any relevant compound of the invention. Thus, for the purposes of this invention, the term also includes prodrugs of the active ingredients described herein.

The term “prodrug” of a relevant active ingredient includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of the active ingredients described herein may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include active ingredients wherein a hydroxyl, amino, sulfhydryl, carboxyl or carbonyl group in a compound of formula I is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

In respect of the following embodiments of the invention, it should be noted that reference to “apomorphine” is also intended to cover aspects of the invention using “an inhaled dopamine agonist formulation”.

### Delivery, and Dosing of Apomorphine

Embodiments of the invention, which may be used alone or be in any combination, include those wherein:

(a) the apomorphine may be in the same composition as the levodopa and/or a dopamine agonist or, particularly, the apomorphine is in a separate composition to a composition comprising levodopa and/or a dopamine agonist;
(b) the apomorphine is delivered by pulmonary inhalation;
(c) the apomorphine is in the form of a composition such as a dry powder composition;
(d) a composition comprising apomorphine comprises at least 5% (e.g. at least about 10%, 20%, 30%, 40%, 50%, 60%, 70%, 80% or 90%) of apomorphine by weight, for example at least about 75%, 85%, 95%, 96%, 97%, 98% or 99% (by weight) apomorphine.
(e) a composition comprising apomorphine further comprises an additive material (e.g. magnesium stearate);
(f) the apomorphine composition further comprises carrier particles made from one or more excipient materials (e.g. inorganic salts, organic salts, other organic compounds sugar or, more particularly, alcohols, polyols and crystalline sugars (such as mannitol, trehalose, melezitose, dextrose or, particularly, lactose);
(g) the carrier particles may have an average particle size between 5 to 1000 μm (e.g. 4 to 500 μm, such as 20 to 200 μm, 30 to 150 μm, 40 to 70 μm, or 60 μm);
(h) the apomorphine composition provides a therapeutic effect with duration of at least 60 minutes (e.g. 60 to 300 minutes, such as 70 to 240 minutes, or, particularly, 80 to 120 minutes);
(i) the maximum daily dose of apomorphine is less than 30 mg (e.g. 27 mg, such as 24.5 mg or, particularly, 22.5 mg);
(j) a dose of apomorphine is provided as a fine particle dose of apomorphine of between 0.5 to 4.5 mg (e.g. 0.5 to 3 mg, such as 1.5 to 3 mg, such as higher than 1.5 mg, but less than 3 mg), for example when measured by a New Generation Impactor (Ph Eur Apparatus at 60 L/min);
(k) the apomorphine can be administered on demand before, or at the onset of an off episode;
(l) when dosed, the Cₘₐₓ of apomorphine is achieved within 10 minutes of administration by inhalation (e.g. between 1 and 5 minutes, such as between 1 and 3 minutes, e.g. 1 and 2 minutes or alternatively the Cₘₐₓ is achieved within 2.5 minutes of administration, such as within 2 minutes of administration, such as within 1.5 minutes of administration, such as within 1 minute of administration.);
(m) the Cₘₐₓ of apomorphine is dose dependent;
(n) the apomorphine provides a therapeutic effect within 10 minutes of administration (e.g. between 2 and 5 minutes);
(o) wherein the apomorphine is administered sequentially, simultaneously or concomitantly with levodopa and/or a dopamine agonist that is not apomorphine;
(p) the apomorphine is used in the absence of an anti-emetic;
(q) suitably the Cₘₐₓ as mentioned herein, is achieved in the majority of patients, such as in at least 50% of patients (e.g. such as at least 60%, such as at least 70%, such as at least 80% of patients);
(r) the concentration of apomorphine decreases to no more than 70% of Cₘₐₓ (e.g. such as no more than 60% of Cₘₐₓ, such as no more than 50% of the Cₘₐₓ, such as no more than 40% of the Cₘₐₓ, e.g. such as no more than 30% of the Cₘₐₓ) in the 4 minutes following Cₘₐₓ;
(s) the concentration of apomorphine decreases to no more than 50% of Cₘₐₓ within 5 minutes of the Cₘₐₓ (e.g. the concentration of apomorphine decreases to no more than 60% of Cₘₐₓ within 7 minutes of the Cₘₐₓ).

In one embodiment, the composition comprises a nominal dose of apomorphine to be administered to a patient, the amount of apomorphine being up to 15 mg, 14 mg, 13 mg, 12 mg, 11 mg, 10 mg, 9 mg, 8 mg, 7 mg, 6 mg or up to 5 mg, in particular, the nominal dose is at least 0.5 mg, 1 mg, 2 mg, 3 mg, 4 mg, 5 mg, 6 mg, 7 mg or 8 mg.

In one embodiment of the present invention, the composition used for treating Parkinson's disease via inhalation comprises a nominal dose of from about 1-8 mg, such as 2.3 mg, 2.4 mg, 3.5 mg, 5 mg, 7.3 mg or 7.7 mg of apomorphine (e.g., apomorphine, apomorphine free base, pharmaceutically acceptable salt(s) or ester(s) thereof, based on the weight of the hydrochloride salt). In one embodiment this nominal dose produces a FPD of 1.5 mg, 2.5 mg, 3.5 mg or 4.5 mg FPD, respectively, of apomorphine.

In one embodiment the nominal dose of apomorphine is able to achieve from 1-5 mg FPD, such as about 1 mg, about 1.5 mg, about 2 mg, about 2.5 mg, about 3 mg, about 3.5 mg, about 4 mg, about 4.5 mg FPD, respectively, of apomorphine, for example when delivered from a passive dry powder inhaler.

In another embodiment of the present invention, the dose of the powder composition delivers, a fine particle dose of from about 400 μg to about 6000 μg of apomorphine (based on the weight of the hydrochloride salt), when measured by a Multistage Liquid Impinger, United States Pharmacopoeia 26, Chapter 601, Apparatus 4 (2003), an Andersen Cascade Impactor or particularly a New Generation Impactor (e.g. Ph Eur Apparatus at 60 L/min). Particularly, the dose delivers a fine particle dose from about 400 to about 5000 μg, such as 1-5 mg, such as 1, 2, 3, 4 or 5 mg, or such as 1.5-4 mg, for example 1.5-3.5 mg, when assessed using such apparatus.

In the context of the present invention, the dose (e.g., in micrograms or milligrams) of apomorphine or its pharmaceutically acceptable salts or esters will be described based upon the weight of the hydrochloride salt (apomorphine hydrochloride). Similarly, the dose of other agents (e.g. levodopa, carbidopa and entacapone) described herein are suitably as defined in the original innovator's prescribing information (e.g. see the prescribing information for Stavelo®, produced by Novartis).

In another embodiment, the composition provides a daily dose, which is the dose administered over a period of 24 hours, of between about 1 and less than 30 mg (e.g. up to 27 mg, such as up to 24.5 mg or, particularly up to 22.5 mg). The daily doses will often be divided up into a number of doses. In particular, the daily dose is between about 1 and about 18 mg (e.g. between about 2 and 16 mg, between about 4 and 14 mg or, particularly between about 5 and 12 mg). These daily doses may be administered at a single instance (usually involving multiple inhalations), but it is expected that the daily dose will be spread out over the 24 hour period with patients receiving, on average, 2-3 separate single administrations, although some patients may receive 5-7 doses (e.g. 6 doses), with a daily extreme of less than 30 mg (e.g. 27 mg, such as 24.5 mg or, particularly, 22.5 mg) in a 24 hour period. It is important to note that the general dose recommendations for apomorphine vary depending on medical authority with respect to the maximum-allowable single dose (i.e. between 6 mg and 10 mg) and the maximum daily dose for apomorphine, with 100 mg and approximately 25 mg being recommended in Europe and the United States of America, respectively.

In another embodiment, the composition allows doses to be administered at regular and frequent intervals, for example intervals of about 60 minutes, about 45 minutes, about 30 minutes, about 20 minutes, about 15 minutes or about 10 minutes, providing maintenance therapy to avoid the patient experiencing off-episodes comparable to the effect of the infusion pump mentioned above. In such an embodiment, the individual doses administered at the chosen intervals will be adjusted to provide a daily dose within safe limits, whilst hopefully providing the patient with adequate relief from symptoms. For example, each individual fine particle dose may provide in the order of about 0.5 mg to about 7 mg apomorphine. A fine particle dose within this range may generally be possible from nominal dose of about 0.8 mg to 11.5 mg. In one embodiment the delivered fine particle dose may be from 0.5 mg to 4.5 mg, from 1.5 mg to 3.0 mg, or more particularly, higher than about 1.5 mg and less than about 3.0 mg from nominal doses of between 0.5-6 mg apomorphine. If the dosing takes place over a period of 11.0 hours (when the patient is awake) and at 60 minute intervals with a fine particle dose of 2 mg, this will provide a daily dose of 22 mg.

In yet another embodiment, there is described a method for treating Parkinson's disease using inhaled apomorphine, wherein between 80 and 95% (e.g. 85 and 93%, such as 87 and 92%) of patients are treatable using a daily dose that is no more than 30 mg (e.g. 27 mg, such as 24.5 mg or, particularly, 22.5 mg). Optionally, the each individual FPD administered is from 0.5 mg to 4.5 mg (e.g. from 1.5 mg to 3.0 mg, or more particularly, higher than about 1.5 mg and less than about 3.0 mg).

In the VR040/2/008 clinical study described herein the majority of patients (i.e. 92%) are able to be effectively treated using a low dose (less than 4 mg (e.g. less than 3.5 mg) nominal dose) of inhaled apomorphine, with side effects that appear to be less frequent and severe than those observed using subcutaneously injected apomorphine (see FIG. 11). Without wishing to be bound by theory, the ability to use a low dose of apomorphine to control off episodes, such as use of less than 4 mg (e.g. less than 3.5 mg) nominal dose, or such as 1-3 mg FPD, may allow for the side effects generally observed with the administration of apomorphine to be minimised.

In one aspect of the invention the use of inhaled apomorphine as described herein provides a superior safety profile and a reduced incidence of typical AEs (i.e. a reduction in one or more of yawning, somnolence, nausea and/or vomiting, dizziness/postural hypotension, rhinorrhoea, hallucination or confusion or, particularly, dyskinesia) when compared to known injected apomorphine treatments for PD as disclosed herein, such as Apokyn®.

Without wishing to be bound by theory, the applicant's believe that a consistent and rapid Tₘₐₓ, as observed following pulmonary administration, could be one of the key reasons why a reduced dyskinesia rate was observed in the VR040/2/008 trial described herein. An “off” episode relates to reduced concentrations of conventional PD therapy. It is at this point that apomorphine will be administered with the aim of “filling the gap” until conventional PD oral treatment takes effect. Due to the variable Tₘₐₓ post subcutaneous (sc) administration there is an increased probability that this will coincide with oral Tₘₐₓ resulting in increased dopamine levels and dyskinesia. Conversely, due to the consistent and rapid Tₘₐₓ obtained following pulmonary inhalation of apomorphine, there appears to be a reduced probability of the resulting Tₘₐₓ coinciding with maximum oral levels resulting in reduced dyskinesia incidence.

In the present clinical studies, more than 80% of patients could be treated using a FPD of 1.5 mg or 2.5 mg, allowing for treatment of a population with only a small number of different fixed dosage units. In a yet still further aspect of the present invention, there is provided a method of treatment of Parkinson's disease in a population using inhaled apomorphine, wherein the whole population of patients is treatable with one of 3 fixed doses of inhaled apomorphine, which provides control of “off” episodes with an acceptable side effect profile for each patient. This is in contrast to the 10 different concentrations of subcutaneously injectable apomorphine currently available.

### Pharmacokinetics

According to one embodiment of the present invention, a composition comprising apomorphine is provided, wherein the administration of the composition by pulmonary inhalation provides a Cₘₐₓ within less than about 10 minutes and particularly within about 5 minutes of administration, with about 2 minutes of administration or even within 1 minute of administration. Most particularly, the Cₘₐₓ is provided within 1 to 5 minutes.

In a further embodiment of the present invention, the administration of the composition by pulmonary inhalation provides a dose dependent Cₘₐₓ.

In one embodiment of the present invention, the dose provides, in vivo, a mean Cₘₐₓ of from about 3.03±0.71 ng/ml to about 11.92±1.17 ng/ml. Alternatively, the Cₘₐₓ achieved is above 1 ng/ml, such as above 2 ng/ml, such as above 3 ng/ml, such as above 4 ng/ml, such as above 5 ng/ml, such as above 6 ng/ml, such as above 7 ng/ml, such as above 8 ng/ml, such as above 9 mg/ml, such as above 10 ng/ml or such as above 11 ng/ml. The upper limit for the Cₘₐₓ may be 100 ng/ml (e.g. 75 ng/ml, such as 50 ng/ml, such as 40 ng/ml, e.g. 35 ng/ml).

In one aspect the concentration of apomorphine decreases to below 20 ng/ml (e.g. 17.5 ng/ml, 15 ng/ml, 12.5 ng/ml, 10 ng/ml, 7.5 ng/ml, 5 ng/ml or 2.5 ng/ml) within 7 minutes of administration.

In one aspect the compositions of apomorphine according to the present invention suitably also have a terminal elimination half-life of between 30 and 70 minutes following pulmonary inhalation. The elimination half life for a dose of apomorphine delivered by pulmonary administration for the treatment of erectile dysfunction has been reported to be approximately 60 min. The elimination half life for a dose of apomorphine delivered by pulmonary administration for the treatment of Parkinson's disease as disclosed herein was approximately 20-60 minutes.

In yet another embodiment, the administration of the composition by pulmonary inhalation provides a therapeutic effect with a duration of at least 45 minutes, particularly at least 60 minutes. In a clinical trial, a mean duration of the therapeutic effect of 75 minutes was observed.

In contrast, in a recent clinical study relating to male erectile dysfunction (VR004/008 Phase IIb study), the majority of patients reported that the duration of action lasted between 2 to 10 minutes, although a couple of patients receiving 310 μg and 430 μg doses did report that the duration of action lasted over 30 minutes.=

The combination of lung pathophysiology and inhaled apomorphine attributes result in rapid and consistent systemic exposure which translates into a rapid and predictable therapeutic effect, both of which are key requirements when considering improved treatments of PD. Particularly, a Tₘₐₓ of as little as 1 minute is observed. The majority of patients achieved conversions (that is, the onset of the therapeutic effect) within 10 minutes of inhaling apomorphine. Some patients reported conversion from the “off” to the “on” state as quickly as 2 minutes after administration of the apomorphine by pulmonary inhalation. This is in contrast to the Tₘₐₓ observed following subcutaneous administration of apomorphine which varies from 10 to 60 minutes and exhibits great patient-to-patient variability.

Thus, a composition comprising apomorphine according to the present invention provides in one embodiment a Cₘₐₓ within 1 to 5 minutes of administration upon administration of the composition by pulmonary inhalation. The Cₘₐₓ is dose dependent. This rapid absorption of the apomorphine upon inhalation suitably allows the administration of these compositions to provide a therapeutic effect in about 10 minutes or less. In some cases, the therapeutic effect is experienced within as little as about 5 minutes, about 2 minutes or even about 1 minute from administration.

Without wishing to be bound by theory, the observed high efficacy and low side effects of the inhaled dopamine agonist (e.g. apomorphine) are thought to be a function of the profile of the dopamine agonist's delivery curve. A high initial Cₘₐₓ is achieved which is considered to initiate the observed therapeutic effects of the drug, followed by rapid decrease of dopamine agonist such that the side effects of dopamine agonist are minimised. Such a profile may allow the effective treatment of diseases other than Parkinson's disease, which are due to dopamine agonist deficiency and the treatment of which uses dopamine agonists, such as certain pituitary tumours and restless legs syndrome.

### Self Dosing

The compositions according to the present invention are for use in providing treatment of the symptoms of Parkinson's disease or for preventing the symptoms altogether, optionally when apomorphine is combined sequentially, simultaneously or concomitantly with other active agents comprising levodopa and/or a dopamine agonist that is not apomorphine. In an embodiment of the invention, the patient is able to administer a dose of apomorphine and to ascertain within a period of no more than about 10 minutes whether that administered dose is sufficient to treat or prevent the symptoms of Parkinson's disease. If a further dose of apomorphine is felt to be necessary, this may be safely administered and the procedure may be repeated until the desired therapeutic effect is achieved.

This self-titration of the apomorphine dose is possible as a result of the rapid onset of the therapeutic effect, the accurate and relatively small dose of apomorphine, the reduced number of available apomorphine dose levels and the low incidence of side effects. It is also important that the mode of administration is painless and convenient; allowing repeated dosing without undue discomfort or inconvenience.

In a particular embodiment, the dose is administered to the patient as a single dose requiring just one inhalation. In one embodiment, the dose is particularly provided in a blister or capsule which is to be dispensed using a dry powder inhaler device. Alternatively, the dose may be dispensed using a pressurised metered dose inhaler (pMDI).

In yet another embodiment, the doses of the apomorphine composition are administered by the patient as needed, that is, when the patient experiences or suspects the onset of an off-period (i.e. the apomorphine is administered before, or at the onset of an off episode). This provides an “on-demand” treatment. In this embodiment, a single effective dose of apomorphine may be administered. Alternatively, multiple smaller doses may be administered sequentially, with the effect of each dosing being assessed by the patient before the next dose is administered. This allows self-titration and optimisation of the dose. As described herein, inhalation of apomorphine can effectively reduce the total time per day spent in off-epidoses compared to apomorphine delivered subcutaneously (e.g. 10 minutes to 2 hours more, such as 15 minutes to 1 hour or, particularly, 20 to 40 minutes less time spent in off-episodes throughout the day) or when compared to a patient taking a conventional dosing regimen (e.g. from 1 to 3 hours, such as 90 minutes to 270 minutes less time spent in off-episodes throughout the day).

### Apomorphine Compositions for Pulmonary Inhalation: Particle Size

In the past, many of the commercially available dry powder inhalers exhibited very poor dosing efficiency, with sometimes as little as 10% of the active agent present in the dose actually being properly delivered to the user so that it can have a therapeutic effect. This low efficiency is simply not acceptable where a high dose of active agent is required for the desired therapeutic effect.

The reason for the lack of dosing efficiency is that a proportion of the active agent in the dose of dry powder tends to be effectively lost at every stage the powder goes through from expulsion from the delivery device to deposition in the lung. For example, substantial amounts of material may remain in the blister/capsule or device. Material may be lost in the throat of the subject due to excessive plume velocity. However, it is frequently the case that a high percentage of the dose delivered exists in particulate forms of aerodynamic diameter in excess of that required.

It is well known that particle impaction in the upper airways of a subject is predicted by the so-called impaction parameter. The impaction parameter is defined as the velocity of the particle multiplied by the square of its aerodynamic diameter. Consequently, the probability associated with delivery of a particle through the upper airways region to the target site of action, is related to the square of its aerodynamic diameter. Therefore, delivery to the lower airways, or the deep lung is dependant on the square of its aerodynamic diameter, and smaller aerosol particles are very much more likely to reach the target site of administration in the user and therefore able to have the desired therapeutic effect.

Particles having aerodynamic diameters of less than 10 μm tend to be deposited in the lung. Particles with an aerodynamic diameter in the range of 2 μm to 5 μm will generally be deposited in the respiratory bronchioles whereas smaller particles having aerodynamic diameters in the range of 0.05 to 3 μm are likely to be deposited in the alveoli. So, for example, high dose efficiency for particles targeted at the alveoli is predicted by the dose of particles below 3 μm, with the smaller particles being most likely to reach that target site.

In one embodiment of the present invention, the composition comprises active particles comprising apomorphine, at least 50%, at least 70% or at least 90% of the active particles having a Mass Median Aerodynamic Diameter (MMAD) of no more than about 10 μm. In another embodiment, at least 50%, at least 70% or at least 90% of the active particles have an MMAD of from about 2 μm to about 5 μm. In yet another embodiment, at least 50%, at least 70% or at least 90% of the active particles have aerodynamic diameters in the range of about 0.05 μm to about 3 μm. In one embodiment of the invention, at least about 90% of the particles of apomorphine have a particle size of 5 μm or less.

Particles having a diameter of less than about 10 μm are, however, thermodynamically unstable due to their high surface area to volume ratio, which provides significant excess surface free energy and encourages particles to agglomerate. In a dry powder inhaler, agglomeration of small particles and adherence of particles to the walls of the inhaler are problems that result in the active particles leaving the inhaler as large agglomerates or being unable to leave the inhaler and remaining adhered to the interior of the device, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung. Consequently, it is essential for the present invention to provide a powder formulation which provides good dosing efficiency and reproducibility, delivering an accurate and predictable dose.

Much work has been done to improve the dosing efficiency of dry powder systems comprising active particles having a size of less than 10 μm, reducing the loss of the pharmaceutically active agent at each stage of the delivery. In the past, efforts to increase dosing efficiency and to obtain greater dosing reproducibility have tended to focus on preventing the formation of agglomerates of fine particles of active agent. Such agglomerates increase the effective size of these particles and therefore prevent them from reaching the lower respiratory tract or deep lung, where the active particles should be deposited in order to have their desired therapeutic effect. Proposed measures have included the use of relatively large carrier particles. The fine particles of active agent tend to become attached to the surfaces of the carrier particles as a result of interparticle forces such as Van der Waals forces. Upon actuation of the inhaler device, the active particles are supposed to detach from the carrier particles and are then present in the aerosol cloud in inhalable form. In addition or as an alternative, the inclusion of additive materials that act as force control agents that modify the cohesion and adhesion between particles has been proposed.

However, where the dose of drug to be delivered is very high, the options for adding materials to the powder composition are limited, especially where at least 70% of the composition is made up of the apomorphine as is particularly disclosed in the present invention. Nevertheless, it is imperative that the dry powder composition exhibit good flow and dispersion properties, to ensure good dosing efficiency.

### Additional Active Ingredients

Further embodiments of the invention include those wherein:

(i) the dopamine agonist, when present, is selected from bromocriptine, pramipexole, ropinirole, rotigotine (e.g. where the daily doses for each are between 2.5 to 100 mg, 0.375 to 6 mg (i.e. 1.5 mg), 0.25 to 24 mg and 2-6 mg per day, respectively);
(ii) the composition of levodopa and/or a dopamine agonist is given orally, transdermally or by infusion (e.g. orally or transdermally);
(iii) the maximum daily dose of levodopa is 1600 mg (e.g. 1500 mg);
(iv) comprises other agents that treat and/or prevent the symptoms of Parkinson's disease;
(v) the composition of levodopa and/or a dopamine agonist further comprises other agents that treat and/or prevent the symptoms of Parkinson's disease (e.g. the composition of levodopa and/or a dopamine agonist further comprises other agents that treat and/or prevent the symptoms of Parkinson's disease may be a single dosage form or multiple dosage forms containing one or more active ingredients);
(vi) the other agents are selected from one or more of further dopamine agonists, mono amine oxidase B inhibitors, aromatic L-amino acid decarboxylase inhibitors, catechol-O-methyltransferase inhibitors, anticholinergics and antimuscarinics (e.g. tolcapone, ipratropium, oxitropium, tiotropium, glycopyrolate, atropine, scopolamine, tropicamide, pirenzepine, diphenhydramine, dimenhydrinate, dicyclomine, flavoxate, oxybutynin, cyclopentolate, trihexyphenidyl, benzhexyl, darifenacin, procyclidine, particularly, difluoromethyldopa, α-methyldopa or, more particulalry, bromocriptine, pramipexole, ropinirole, rotigotine, carbidopa, benserazide, selegiline, rasagiline, entacapone);
(vii) suitable the daily doses for bromocriptine (2.5 to 100 mg), pramipexole (0.375 to 6 mg (i.e. 1.5 mg)), ropinirole (0.25 to 24 mg), rotigotine (2-6 mg), carbidopa (12.5 to 300 mg), benserazide (25 to 200 mg), selegiline (1.25 to 2.5 mg), rasagiline (1 mg), entacapone (200 to 1600 mg), tolcapone (100 to 600 mg), ipratropium (17 to 84 μg), tiotropium (18 to 36 μg), glycopyrolate (2 to 8 mg), atropine (7.5 to 20 mg), scopolamine (0.4 to 0.8 mg), diphenhydramine (10 to 400 mg), dimenhydrinate (50 to 400 mg), dicyclomine (30 to 160 mg), flavoxate (300 to 800 mg), oxybutynin (10 to 15 mg), trihexyphenidyl (3 to 6 mg), benzhexyl (1 to 4 mg), darifenacin (7.5 to 15 mg) and procyclidine (0.4 to 0.6 mg) are as indicated by parenthesis;
(viii) the levodopa is provided in combination with carbidopa and, optionally, entacapone;
(ix) the composition of levodopa and/or a dopamine agonist is taken as part of a recognised therapeutic dosing regimen for the treatment of Parkinson's disease;
(x) levodopa and/or dopamine agonist (optionally comprising other active agents) may be administered sequentially, simultaneously or concomitantly with apomorphine.

In accordance with the invention, apomorphine may be administered alone (i.e. as a monotherapy). In alternative embodiments of the invention, however, apomorphine may be administered in combination with another therapeutic agent (e.g. another therapeutic agent for the treatment of PD).

When used herein, the term “another therapeutic agent” includes references to one or more (e.g. one) therapeutic agents that are known to be useful for (e.g. that are known to be effective in) the treatment of Parkinson's disease.

Particular other therapeutic agents that may be mentioned include, for example, levodopa (L-DOPA), dopamine agonists (e.g. pramipexole, ropinirole or rotigotine), monoamine oxidase B inhibitors (e.g. selegiline or rasagiline), catechol O-methyl transferase inhibitors (e.g. entacapone or tolcapone), amantadine, acetylcholinesterase inhibitors (e.g. donepezil, rivastigmine or galantamine) and glutamate inhibitors (e.g. memantine) and other agents as described herein.

When used herein, the term “administered sequentially, simultaneously or concomitantly” includes references to:
administration of separate pharmaceutical formulations (one containing the apomorphine and one or more others containing the one or more other therapeutic agents); and
administration of a single pharmaceutical formulation containing the apomorphine and the other therapeutic agent(s).

In a particular embodiment, when levodopa is to be administered by pulmonary inhalation, the apomorphine and levodopa are delivered from different receptacles.

The other active agents described herein (i.e. those not apomorphine) may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, transdermal, intravenous, cutaneous, subcutaneous, nasal, intramuscular or intraperitoneal administration. Yet more particular modes of administration that may be mentioned include oral and transdermal administration.

The other active agents described herein will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pa. (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of the other active agents described herein in any pharmaceutical formulation used in accordance with the present invention will depend on various factors, such as the severity of the condition to be treated, the particular patient to be treated, as well as the compound(s) which is/are employed. In any event, the amount the other active agents described herein in the formulation may be determined routinely by the skilled person.

For example, a solid oral composition such as a tablet or capsule may contain from 1 to 99% (w/w) active ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) active ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the disorder, and the patient, to be treated, as well as the route of administration, the other active agents described herein may be administered at varying therapeutically effective doses to a patient in need thereof.

However, the dose administered to a mammal, particularly a human, in the context of the present invention should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Administration may be continuous or intermittent (e.g. by bolus injection). The dosage may also be determined by the timing and frequency of administration. In the case of oral or parenteral administration the dosage can vary from about 0.01 mg to about 2000 mg per day of the other active agents described herein.

In any event, the medical practitioner, or other skilled person, will be able to determine routinely the actual dosage, which will be most suitable for an individual patient. The above-mentioned dosages are exemplary of the average case; there can, of course, be individual instances where higher or lower dosage ranges are merited, and such are within the scope of this invention.

The aspects of the invention described herein (e.g. the above-mentioned compounds, combinations, methods and uses) may have the advantage that, in the treatment of the conditions described herein, they may be more convenient for the physician and/or patient than, be more efficacious than, be less toxic than, have better selectivity over, have a broader range of activity than, be more potent than, produce fewer side effects than, or may have other useful pharmacological properties over, similar compounds, combinations, methods (treatments) or uses known in the prior art for use in the treatment of those conditions or otherwise.

Side effects that may be mentioned in this respect include side effects caused by the overstimulation of dopamine receptors in the peripheral nervous system (such as dyskinesia, los of sleep, yawning, somnolence, nausea/vomiting, dizziness/postural hypotension, rhinorrhoea and hallucination or confusion).

### Additives

The tendency of fine particles to agglomerate means that the FPF of a given dose can be highly unpredictable and a variable proportion of the fine particles will be administered to the lung, or to the correct part of the lung, as a result. This is observed, for example, in formulations comprising pure drug in fine particle form. Such formulations exhibit poor flow properties and poor FPF.

In an attempt to improve this situation and to provide a consistent FPF and FPD, dry powder compositions according to the present invention may include additive material which is an anti-adherent material and reduces cohesion between the particles in the composition.

The additive material is selected to reduce the cohesion between particles in the dry powder composition. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles may return to the form of small individual particles or agglomerates of small numbers of particles which are capable of reaching the lower lung.

The additive material may be in the form of particles which tend to adhere to the surfaces of the active particles, as disclosed in WO 1997/03649. Alternatively, the additive material may be coated on the surface of the active particles by, for example a co-milling method as disclosed in WO 2002/43701.

Particularly, the additive material is an anti-adherent material and it will tend to reduce the cohesion between particles and will also prevent fine particles becoming attached to surfaces within the inhaler device. Advantageously, the additive material is an anti-friction agent or glidant and will give the powder formulation better flow properties in the inhaler. The additive materials used in this way may not necessarily be usually referred to as anti-adherents or anti-friction agents, but they will have the effect of decreasing the cohesion between the particles or improving the flow of the powder. The additive materials are sometimes referred to as force control agents (FCAs) and they usually lead to better dose reproducibility and higher FPFs.

Therefore, an additive material or FCA, as used herein, is a material whose presence on the surface of a particle can modify the adhesive and cohesive surface forces experienced by that particle, in the presence of other particles and in relation to the surfaces that the particles are exposed to. In general, its function is to reduce both the adhesive and cohesive forces.

The reduced tendency of the particles to bond strongly, either to each other or to the device itself, not only reduces powder cohesion and adhesion, but can also promote better flow characteristics. This leads to improvements in the dose reproducibility because it reduces the variation in the amount of powder metered out for each dose and improves the release of the powder from the device. It also increases the likelihood that the active material, which does leave the device, will reach the lower lung of the patient.

It is favourable for unstable agglomerates of particles to be present in the powder when it is in the inhaler device. As indicated above, for a powder to leave an inhaler device efficiently and reproducibly, the particles of such a powder should be large, particularly larger than about 40 μm. Such a powder may be in the form of either individual particles having a size of about 40 μm or larger and/or agglomerates of finer particles, the agglomerates having a size of about 40 μm or larger. The agglomerates formed can have a size of 100 μm or 200 μm and, depending on the type of device used to dispense the formulation, the agglomerates may be as much as about 1000 μm. With the addition of the additive material, those agglomerates are more likely to be broken down efficiently in the turbulent airstream created on inhalation. Therefore, the formation of unstable or “soft” agglomerates of particles in the powder may be favoured compared with a powder in which there is substantially no agglomeration. Such unstable agglomerates are stable whilst the powder is inside the device but are then disrupted and broken up upon inhalation.

It is particularly advantageous for the additive material to comprise an amino acid. Amino acids have been found to give, when present as additive material, high respirable fraction of the active material and also good flow properties of the powder. A particular amino acid that may be mentioned is leucine, in particular L-leucine, di-leucine and tri-leucine. Although the L-form of the amino acids is generally used, the D- and DL-forms may also be used. The additive material may comprise one or more of any of the following amino acids: aspartame, leucine, isoleucine, lysine, valine, methionine, cysteine, and phenylalanine. Additive materials may also include, for example, metal stearates such as magnesium stearate, phospholipids, lecithin, colloidal silicon dioxide and sodium stearyl fumarate, and are described more fully in WO 1996/23485, which is hereby incorporated by reference.

Advantageously, the powder includes at least 80%, particularly at least 90% by weight of active ingredients (e.g. apomorphine (or its pharmaceutically acceptable salts), optionally comprising other active ingredients, such as levodopa and carbidopa) based on the weight of the powder. The optimum amount of additive material will depend upon the precise nature of the additive and the manner in which it is incorporated into the composition. In some embodiments, the powder advantageously includes not more than 8%, more advantageously not more than 5% by weight of additive material based on the weight of the powder. As indicated above, in some cases it will be advantageous for the powder to contain about 1% by weight of additive material. In other embodiments, the additive material or FCA may be provided in an amount from about 0.1% to about 10% by weight, and particularly from about 0.15% to 5%, most particularly from about 0.5% to about 2%.

When the additive material is micronised leucine or lecithin, it is particularly provided in an amount from about 0.1% to about 10% by weight. Particularly, the additive material comprises from about 3% to about 7%, particularly about 5%, of micronised leucine. Particularly, at least 95% by weight of the micronised leucine has a particle diameter of less than 150 μm, particularly less than 100 μm, and most particularly less than 50 μm. Particularly, the mass median diameter of the micronised leucine is less than 10 μm.

If magnesium stearate or sodium stearyl fumarate is used as the additive material, it is particularly provided in an amount from about 0.05% to about 10%, from about 0.15% to about 5%, from about 0.25% to about 3%, or from about 0.5% to about 2.0% depending on the required final dose.

In a further attempt to improve extraction of the dry powder from the dispensing device and to provide a consistent FPF and FPD, dry powder compositions according to the present invention may include particles of an inert excipient material, which act as carrier particles. These carrier particles are mixed with fine particles of active material and any additive material which is present. Rather than sticking to one another, the fine active particles tend to adhere to the surfaces of the carrier particles whilst in the inhaler device, but are supposed to release and become dispersed upon actuation of the dispensing device and inhalation into the respiratory tract, to give a fine suspension.

The inclusion of carrier particles is less attractive where very large doses of active agent are to be delivered, as they tend to significantly increase the volume of the powder composition. Nevertheless, in some embodiments of the present invention, the compositions include carrier particles. In such an embodiment, the composition comprises at least about 10% (by weight) of the active ingredient(s) (i.e. apomorphine alone, or optionally in combination with one or more active ingredients), or at least about 15%, 17%, or 18% or 18.5% (by weight) of the active ingredient(s) (i.e. apomorphine alone, or optionally in combination with one or more active ingredients). More particularly, the carrier particles are present in small amount, such as no more than 90% (e.g. 85%, 83% or, more particularly 80%) by weight of the total composition, in which the total apomorphine and magnesium stearate content would be about 18.5 and 1.5% by weight, respectively.

Carrier particles may be of any acceptable inert excipient material or combination of materials. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles comprise a polyol. In particular, the carrier particles may be particles of crystalline sugar, for example mannitol, trehalose, melezitose, dextrose or lactose. Most particularly, the carrier particles are composed of lactose.

Thus, in one embodiment of the present invention, the composition comprises active particles comprising apomorphine and carrier particles. The carrier particles may have an average particle size of from about 5 to about 1000 μm, from about 4 to about 40 μm, from about 60 to about 200 μm, or from 150 to about 1000 μm. Other useful average particle sizes for carrier particles are about 20 to about 30 μm or from about 40 to about 70 μm.

In an alternate embodiment, the carrier particles are present in small amount, such as no more than 50% (e.g. 60%, 70% or, more particularly, 80%) by weight of the total composition, in which the total apomorphine and magnesium stearate content, by weight, would be 18 and 2% respectively. As the amount of carrier in these formulations changes, the amounts of additive and apomorphine will also change, but the ratio of these constituents particularly remains approximately 1:9 to about 1:13.

In an alternate embodiment, the formulation does not contain carrier particles and comprises apomorphine and additive, such as at least 30% (e.g. 60%, 80%, 90%, 95% or, more particularly, 97%) by weight of the total composition comprises of pharmaceutically active agent. The active agent may be apomorphine alone or it may be a combination of the apomorphine and an anti-emetic drug or another drug which would benefit Parkinson's disease patients. The remaining components may comprise one or more additive materials, such as those discussed above.

In a further embodiment the formulation may contain carrier particles and comprises of the active ingredient(s) (i.e. apomorphine alone, or optionally in combination with one or more active ingredients) and additive, such as at least 30% (e.g. 60%, 80%, 90%, 95% or, more particularly, 97%) by weight of the total composition comprises the pharmaceutically active agent and wherein the remaining components comprise additive material and larger particles. The larger particles provide the dual action of acting as a carrier and facilitating powder flow.

In a particular embodiment, the composition comprises apomorphine (30% w/w) and lactose having an average particles size of 45-65 μm.

The compositions comprising active ingredient(s) (i.e. apomorphine alone, or optionally in combination with one or more active ingredients) and carrier particles may further include one or more additive materials. The additive material may be in the form of particles which tend to adhere to the surfaces of the active particles, as disclosed in WO 1997/03649. Alternatively, the additive material may be coated on the surface of the active particles by, for example a co-milling method as disclosed in WO 2002/43701 or on the surfaces of the carrier particles, as disclosed in WO 2002/00197.

In one embodiment, the additive is coated onto the surface of the carrier particles. This coating may be in the form of particles of additive material adhering to the surfaces of the carrier particles (by virtue of interparticle forces such as Van der Waals forces), as a result of a blending of the carrier and additive. Alternatively, the additive material may be smeared over and fused to the surfaces of the carrier particles, thereby forming composite particles with a core of inert carrier material and additive material on the surface. For example, such fusion of the additive material to the carrier particles may be achieved by co-jet milling particles of additive material and carrier particles. In some embodiments, all three components of the powder (active, carrier and additive) are processed together so that the additive becomes attached to or fused to both the carrier particles and the active particles. In one illustrative embodiment, the compositions include an additive material, such as magnesium stearate (up to 10% w/w) or leucine, said additive being jet-milled with the particles of apomorphine and/or with the lactose.

In a particular embodiment described herein, the formulation comprises one or more of:
(a) an additive material (e.g. magnesium stearate); and
(b) a carrier (e.g. lactose fines).

In certain embodiments of the present invention, the apomorphine formulation is a “carrier free” formulation, which includes only the apomorphine or its pharmaceutically acceptable salts or esters and one or more additive materials.

Advantageously, in these “carrier free” formulations, at least 90% by weight of the particles of the powder have a particle size less than 63 μm, particularly less than 30 μm and more particularly less than 10 μm. As indicated above, the size of the apomorphine (or its pharmaceutically acceptable salts) particles of the powder should be within the range of about from 0.1 μm to 5 μm for effective delivery to the lower lung. Where the additive material is in particulate form, it may be advantageous for these additive particles to have a size outside the preferred range for delivery to the lower lung.

The powder includes at least 60% by weight of the apomorphine or a pharmaceutically acceptable salt or ester thereof based on the weight of the powder. Advantageously, the powder comprises at least 70%, or at least 80% by weight of apomorphine or a pharmaceutically acceptable salt or ester thereof based on the weight of the powder. Most advantageously, the powder comprises at least 90%, at least 95%, or at least 97% by weight of apomorphine or a pharmaceutically acceptable salt or ester thereof based on the weight of the powder. It is believed that there are physiological benefits in introducing as little powder as possible to the lungs, in particular material other than the active ingredient to be administered to the patient. Therefore, the quantities in which the additive material is added are particularly as small as possible. Most particularly the powder, therefore, would comprise more than 99% by weight of apomorphine or a pharmaceutically acceptable salt or ester thereof.

Apomorphine can exist in a free base form or as an acid addition salt. For the purposes of the present invention apomorphine hydrochloride and the apomorphine free base forms are particularly mentioned herein, but other pharmacologically acceptable forms of apomorphine can also be used. The term “apomorphine” as used herein includes the free base form of this compound as well as the pharmacologically acceptable salts or esters thereof. In a particular embodiment, at least some of the apomorphine is in amorphous form. A formulation containing amorphous apomorphine will possess particular dissolution characteristics. A stable form of amorphous apomorphine may be prepared using suitable sugars such as trehalose and melezitose.

In addition to the hydrochloride salt, other acceptable acid addition salts include the hydrobromide, the hydroiodide, the bisulfate, the phosphate, the acid phosphate, the lactate, the citrate, the tartrate, the salicylate, the succinate, the maleate, the gluconate, and the like.

As used herein, the term “pharmaceutically acceptable esters” of apomorphine refers to esters formed with one or both of the hydroxyl functions at positions 10 and 11, and which hydrolyse in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butryates, acrylates and ethyl succinates.

Any of the compositions disclosed herein may be formulated using the apomorphine free base. Alternatively, apomorphine hydrochloride hemi-hydrate is also a form that may be used.

### Preparing Dry Powder Inhaler Formulations

Where the compositions of the present invention include an additive material, the manner in which this is incorporated will have a significant impact on the effect that the additive material has on the powder performance, including the FPF and FPD.

In one embodiment, the compositions according to the present invention are prepared by simply blending particles of the active ingredient(s) (i.e. apomorphine alone, or optionally in combination with one or more active ingredients) of a selected appropriate size with particles of additive material and/or carrier particles. The powder components may be blended by a gentle mixing process, for example in a tumble mixer such as a Turbula (trade mark). In such a gentle mixing process, there is generally substantially no reduction in the size of the particles being mixed. In addition, the powder particles do not tend to become fused to one another, but they rather agglomerate as a result of cohesive forces such as Van der Waals forces. These loose or unstable agglomerates readily break up upon actuation of the inhaler device used to dispense the composition.

### Compressive Milling Processes

In an alternative process for preparing the compositions according to the present invention, the powder components undergo a compressive milling process, such as processes termed mechanofusion (also known as ‘Mechanical Chemical Bonding’) and cyclomixing.

As the name suggests, mechanofusion is a dry coating process designed to mechanically fuse a first material onto a second material. It should be noted that the use of the terms “mechanofusion” and “mechanofused” are supposed to be interpreted as a reference to a particular type of milling process, but not a milling process performed in a particular apparatus. The compressive milling processes work according to a different principle to other milling techniques, relying on a particular interaction between an inner element and a vessel wall, and they are based on providing energy by a controlled and substantial compressive force. The process works particularly well where one of the materials is generally smaller and/or softer than the other.

The fine active particles and additive particles are fed into the vessel of a mechanofusion apparatus (such as a Mechano-Fusion system (Hosokawa Micron Ltd) or the Nobilta or Nanocular apparatus, where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the additive particles around the core particle to form a coating. The energy is generally sufficient to break up agglomerates and some degree of size reduction of both components may occur.

These mechanofusion and cyclomixing processes apply a high enough degree of force to separate the individual particles of active material and to break up tightly bound agglomerates of the active particles such that effective mixing and effective application of the additive material to the surfaces of those particles is achieved. An especially desirable aspect of the processes is that the additive material becomes deformed in the milling and may be smeared over or fused to the surfaces of the active particles.

However, in practice, these compression milling processes produce little or no size reduction of the drug particles, especially where they are already in a micronised form (i.e. <10 μm). The only physical change which may be observed is a plastic deformation of the particles to a rounder shape.

### Other Milling Procedures

The process of milling may also be used to formulate the dry powder compositions according to the present invention. The manufacture of fine particles by milling can be achieved using conventional techniques. In the conventional use of the word, “milling” means the use of any mechanical process which applies sufficient force to the particles of active material that it is capable of breaking coarse particles (for example, particles with a MMAD greater than 100 μm) down to fine particles (for example, having a MMAD not more than 50 μm). In the present invention, the term “milling” also refers to deagglomeration of particles in a formulation, with or without particle size reduction. The particles being milled may be large or fine prior to the milling step. A wide range of milling devices and conditions are suitable for use in the production of the compositions of the inventions. The selection of appropriate milling conditions, for example, intensity of milling and duration, to provide the required degree of force will be within the ability of the skilled person.

Impact milling processes may be used to prepare compositions comprising apomorphine according to the present invention, with or without additive material. Such processes include ball milling and the use of a homogenizer.

Ball milling is a suitable milling method for use in the prior art co-milling processes. Centrifugal and planetary ball milling are especially particular methods.

Alternatively, a high pressure homogeniser may be used in which a fluid containing the particles is forced through a valve at high pressure producing conditions of high shear and turbulence. Shear forces on the particles, impacts between the particles and machine surfaces or other particles, and cavitation due to acceleration of the fluid may all contribute to the fracture of the particles. Suitable homogenisers include EmulsiFlex high pressure homogenisers which are capable of pressures up to 4000 bar, Niro Soavi high pressure homogenisers (capable of pressures up to 2000 bar), and Microfluidics Microfluidisers (maximum pressure 2750 bar). The milling process can be used to provide the microparticles with mass median aerodynamic diameters as specified above. Homogenisers may be more suitable than ball mills for use in large scale preparations of the composite active particles.

The milling step may, alternatively, involve a high energy media mill or an agitator bead mill, for example, the Netzsch high energy media mill, or the DYNO-mill (Willy A. Bachofen AG, Switzerland).

If a significant reduction in particle size is also required, co-jet milling is used particularly, as disclosed in the earlier patent application published as WO 2005/025536. The co-jet milling process can result in composite active particles with low micron or sub-micron diameter, and these particles exhibit particularly good FPF and FPD, even when dispensed using a passive DPI.

The milling processes apply a high enough degree of force to break up tightly bound agglomerates of fine or ultra-fine particles, such that effective mixing and effective application of the additive material to the surfaces of those particles is achieved.

These impact processes create high-energy impacts between media and particles or between particles. In practice, while these processes are good at making very small particles, it has been found that neither the ball mill nor the homogenizer was particularly effective in producing dispersion improvements in resultant drug powders in the way observed for the compressive process. It is believed that the second impact processes are not as effective in producing a coating of additive material on each particle.

Conventional methods comprising co-milling active material with additive materials (as described in WO 2002/43701) result in composite active particles which are fine particles of active material with an amount of the additive material on their surfaces. The additive material is particularly in the form of a coating on the surfaces of the particles of active material. The coating may be a discontinuous coating. The additive material may be in the form of particles adhering to the surfaces of the particles of active material. Co-milling or co-micronising particles of active agent and particles of additive (FCA) or excipient will result in the additive or excipient becoming deformed and being smeared over or fused to the surfaces of fine active particles, producing composite particles made up of both materials. These resultant composite active particles comprising an additive have been found to be less cohesive after the milling treatment.

At least some of the composite active particles may be in the form of agglomerates. However, when the composite active particles are included in a pharmaceutical composition, the additive material promotes the dispersal of the composite active particles on administration of that composition to a patient, via actuation of an inhaler.

Milling may also be carried out in the presence of a material which can delay or control the release of the active agent.

The co-milling or co-micronising of active and additive particles may involve compressive type processes, such as mechanofusion, cyclomixing and related methods such as those involving the use of a Hybridiser or the Nobilta. The principles behind these processes are distinct from those of alternative milling techniques in that they involve a particular interaction between an inner element and a vessel wall, and in that they are based on providing energy by a controlled and substantial compressive force, particularly compression within a gap of predetermined width.

In one embodiment, if required, the microparticles produced by the milling step can then be formulated with an additional excipient. This may be achieved by a spray drying process, e.g. co-spray drying with excipients. In this embodiment, the particles are suspended in a solvent and co-spray dried with a solution or suspension of the additional excipient. Particular additional excipients include trehalose, melezitose and other polysaccharides. Additional pharmaceutical effective excipients may also be used.

In another embodiment, the powder compositions are produced using a multi-step process. Firstly, the materials are milled or blended. Next, the particles may be sieved, prior to undergoing mechanofusion. A further optional step involves the addition of carrier particles. The mechanofusion step is thought to “polish” the composite active particles, further rubbing the additive material into the active particles. This allows one to enjoy the beneficial properties afforded to particles by mechanofusion, in combination with the very small particles sizes made possible by the jet milling.

The reduction in the cohesion and adhesion between the active particles can lead to equivalent performance with reduced agglomerate size, or even with individual particles.

### High Shear Blending

Scaling up of pharmaceutical product manufacture often requires the use one piece of equipment to perform more than one function. An example of this is the use of a mixer-granulator which can both mix and granulate a product thereby removing the need to transfer the product between pieces of equipment. In so doing, the opportunity for powder segregation is minimised. High shear blending often uses a high-shear rotor/stator mixer (HSM), which has become used in mixing applications. Homogenizers or “high shear material processors” develop a high pressure on the material whereby the mixture is subsequently transported through a very fine orifice or comes into contact with acute angles. The flow through the chambers can be reverse flow or parallel flow depending on the material being processed. The number of chambers can be increased to achieve better performance. The orifice size or impact angle may also be changed for optimizing the particle size generated. Particle size reduction occurs due to the high shear generated by the high shear material processors while it passes through the orifice and the chambers. The ability to apply intense shear and shorten mixing cycles gives these mixers broad appeal for applications that require agglomerated powders to be evenly blended. Furthermore conventional HSMs may also be widely used for high intensity mixing, dispersion, disintegration, emulsification and homogenization.

It is well known to those skilled in the production of powder formulations that small particles, even with high-power, high-shear, mixers a relatively long period of “aging” is required to obtain complete dispersion, and this period is not shortened appreciably by increases in mixing power, or by increasing the speed of rotation of the stirrer so as to increase the shear velocity. High shear mixers can also be used if the auto-adhesive properties of the drug particles are so that high shear forces are required together with use of a force-controlling agent for forming a surface-energy-reducing particulate coating or film.

### Spray Drying and Ultrasonic Nebulisers

Spray drying may be used to produce particles of inhalable size comprising the apomorphine. The spray drying process may be adapted to produce spray-dried particles that include the active agent and an additive material which controls the agglomeration of particles and powder performance. The spray drying process may also be adapted to produce spray-dried particles that include the active agent dispersed or suspended within a material that provides the controlled release properties. Furthermore the dispersal or suspension of the active material within an excipient material may impart further stability to the active compounds. In a particular embodiment the apomorphine may reside primarily in the amorphous state. A formulation containing amorphous apomorphine will possess paricular dissolution characteristics. This would be possible in that particles are suspended in a sugar glass which could be either a solid solution or a solid dispersion. Particular additional excipients include trehalose, melezitose and other polysaccharides.

Spray drying is a well-known and widely used technique for producing particles of active material of inhalable size. Conventional spray drying techniques may be improved so as to produce active particles with enhanced chemical and physical properties so that they perform better when dispensed from a DPI than particles formed using conventional spray drying techniques. Such improvements are described in detail in the earlier patent application published as WO 2005/025535.

In particular, it is disclosed that co-spray drying an active agent with an FCA under specific conditions can result in particles with excellent properties which perform extremely well when administered by a DPI for inhalation into the lung.

It has been found that manipulating or adjusting the spray drying process can result in the FCA being largely present on the surface of the particles. That is, the FCA is concentrated at the surface of the particles, rather than being homogeneously distributed throughout the particles. This clearly means that the FCA will be able to reduce the tendency of the particles to agglomerate. This will assist the formation of unstable agglomerates that are easily and consistently broken up upon actuation of a DPI.

It has been found that it may be advantageous to control the formation of the droplets in the spray drying process, so that droplets of a given size and of a narrow size distribution are formed. Furthermore, controlling the formation of the droplets can allow control of the air flow around the droplets which, in turn, can be used to control the drying of the droplets and, in particular, the rate of drying. Controlling the formation of the droplets may be achieved by using alternatives to the conventional 2-fluid nozzles, especially avoiding the use of high velocity air flows.

In particular, it is preferred to use a spray drier comprising a means for producing droplets moving at a controlled velocity and of a predetermined droplet size. The velocity of the droplets is particularly controlled relative to the body of gas into which they are sprayed. This can be achieved by controlling the droplets' initial velocity and/or the velocity of the body of gas into which they are sprayed, for example by using an ultrasonic nebuliser (USN) to produce the droplets. Alternative nozzles such as electrospray nozzles or vibrating orifice nozzles may be used.

In one embodiment, a USN is used to form the droplets in the spray mist. USNs use an ultrasonic transducer which is submerged in a liquid. The ultrasonic transducer (a piezoelectric crystal) vibrates at ultrasonic frequencies to produce the short wavelengths required for liquid atomisation. In one common form of USN, the base of the crystal is held such that the vibrations are transmitted from its surface to the nebuliser liquid, either directly or via a coupling liquid, which is usually water. When the ultrasonic vibrations are sufficiently intense, a fountain of liquid is formed at the surface of the liquid in the nebuliser chamber. Droplets are emitted from the apex and a “fog” emitted.

Whilst USNs are known, these are conventionally used in inhaler devices, for the direct inhalation of solutions containing drug, and they have not previously been widely used in a spray drying apparatus. It has been discovered that the use of such a nebuliser in spray drying has a number of important advantages and these have not previously been recognised. The particular USNs control the velocity of the particles and therefore the rate at which the particles are dried, which in turn affects the shape and density of the resultant particles. The use of USNs also provides an opportunity to perform spray drying on a larger scale than is possible using conventional spray drying apparatus with conventional types of nozzles used to create the droplets, such as 2-fluid nozzles.

The attractive characteristics of USNs for producing fine particle dry powders include: low spray velocity; the small amount of carrier gas required to operate the nebulisers; the comparatively small droplet size and narrow droplet size distribution produced; the simple nature of the USNs (the absence of moving parts which can wear, contamination, etc.); the ability to accurately control the gas flow around the droplets, thereby controlling the rate of drying; and the high output rate which makes the production of dry powders using USNs commercially viable in a way that is difficult and expensive when using a conventional two-fluid nozzle arrangement.

USNs do not separate the liquid into droplets by increasing the velocity of the liquid. Rather, the necessary energy is provided by the vibration caused by the ultrasonic nebuliser.

Further embodiments, may employ the use of ultrasonic nebuliser (USN), rotary atomisers or electrohydrodynamic (EHD) atomizers to generate the particles.

### Delivery Devices

The inhalable compositions in accordance with the present invention are particularly administered via a dry powder inhaler (DPI), but can also be administered via a pressurized metered dose inhaler (pMDI), or even via a nebulised system.

In a dry powder inhaler, the dose to be administered is stored in the form of a non-pressurized dry powder and, on actuation of the inhaler, the particles of the powder are expelled from the device in the form of a cloud of finely dispersed particles that may be inhaled by the patient.

Dry powder inhalers can be “passive” devices in which the patient's breath is the only source of gas which provides a motive force in the device. Examples of “passive” dry powder inhaler devices include the Rotahaler and Diskhaler (GlaxoSmithKline), the Monohaler (MIAT), the GyroHaler (Trade Mark) (Vectura) the Turbohaler (Astra-Draco) and Novolizer (Trade Mark) (Viatris GmbH). Alternatively, “active” devices may be used, in which a source of compressed gas or alternative energy source is used. Examples of suitable active devices include Aspirair (Trade Mark) (Vectura) and the active inhaler device produced by Nektar Therapeutics (as covered by U.S. Pat. No. 6,257,233).

It is generally considered that different compositions perform differently when dispensed using passive and active type inhalers. Passive devices create less turbulence within the device and the powder particles are moving more slowly when they leave the device. This leads to some of the metered dose remaining in the device and, depending on the nature of the composition, less deagglomeration upon actuation. However, when the slow moving cloud is inhaled, less deposition in the throat is often observed. In contrast, active devices create more turbulence when they are activated. This results in more of the metered dose being extracted from the blister or capsule and better deagglomeration as the powder is subjected to greater shear forces. However, the particles leave the device moving faster than with passive devices and this can lead to an increase in throat deposition.

It has been surprisingly found that the compositions of the present invention with their high proportion of apomorphine perform well when dispensed using both active and passive devices. Whilst there tends to be some loss along the lines predicted above with the different types of inhaler devices, this loss is minimal and still allows a substantial proportion of the metered dose of apomorphine to be deposited in the lung. Once it reaches the lung, the apomorphine is rapidly absorbed and exhibits excellent bioavailability.

Particularly, “active” dry powder inhalers that may be mentioned herein are referred to as Aspirair® inhalers and are described in more detail in WO 2001/00262, WO 2002/07805, WO 2002/89880 and WO 2002/89881, the contents of which are hereby incorporated by reference. Particular “passive” dry powder inhalers that may be mentioned herein are “passive” dry powder inhalation devices that are described in WO 2010/086285. It should be appreciated, however, that the compositions of the present invention can be administered with either passive or active inhaler devices.

In an alternative embodiment, the composition is a solution or suspension, which is dispensed using a pressurised metered dose inhaler (pMDI). The composition according to this embodiment can comprise the dry powder composition discussed above, mixed with or dissolved in a liquid propellant such as HFA 134a or HFA 227.

In a yet further embodiment, the composition is a solution or suspension and is administered using a pressurised metered dose inhaler (pMDI), a nebuliser or a soft mist inhaler. Examples of suitable devices include pMDIs such as Modulite® (Chiesi), SkyeFine™ and SkyeDry™ (SkyePharma). Nebulisers such as Porta-Neb®, Inquaneb™ (Pan) and Aquilon™, and soft mist inhalers such as eFlow™ (Pan), Aerodose™ (Aerogen), Respimat® Inhaler (Boehringer Ingelheim GmbH), AERx® Inhaler (Aradigm) and Mystic™ (Ventaira Pharmaceuticals, Inc.).

Where the composition is to be dispensed using a pMDI, the composition comprising apomorphine optionally further comprises a propellant (i.e. further comprises a propellant). In embodiments of the present invention, the propellant is CFC-12 or an ozone-friendly, non-CFC propellant, such as 1,1,1,2-tetrafluoroethane (HFC-134a), 1,1,1,2,3,3,3-heptafluoropropane (HFC-227), HCFC-22 (difluororchloromethane), HFA-152 (difluoroethane and isobutene) or combinations thereof. Such formulations may require the inclusion of a polar surfactant such as polyethylene glycol, diethylene glycol monoethyl ether, polyoxyethylene sorbitan monolaurate, polyoxyethylene sorbitan monooleate, propoxylated polyethylene glycol, and polyoxyethylene lauryl ether for suspending, solubilizing, wetting and emulsifying the active agent and/or other components, and for lubricating the valve components of the MDI.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### The VR040/2/008 Clinical Study

A double-blind, randomized, placebo-controlled phase II clinical trial was undertaken to evaluate the efficacy and safety of a treatment according to the invention. A dry powder formulation of apomorphine was administered using the Aspirair® “active” dry powder inhaler (DPI) to allow delivery with a high lung penetration and low variability. The objective of the clinical study was to identify optimal doses of an inhaled dry powder formulation of apomorphine for future evaluation, to determine its efficacy in controlling the “on-off” and “wearing-off” effects associated with fluctuating idiopathic Parkinson's disease (PD) and to determine its safety and tolerability.

The total study period was approximately 18 months from the start of screening to last patient, last visit. The study schedule included a screening period, an in-clinic dosing titration period and an at-home dosing period. All subjects were provided with domperidone (or an equivalent anti-emetic) to use for the duration of the study participation.

At least fifty-five patients, diagnosed with fluctuating idiopathic PD from 15 centres in three countries were enrolled into the study. Evaluable patients were randomly assigned to study treatment in the ratio of 2 active to 1 placebo (forty-five in the active treatment group and fifteen in the placebo group).

Enrolled patients were male and female, aged thirty to ninety years with a diagnosis of PD of at least 5 years duration; fulfilled Steps 1 and 2 of the United Kingdom (UK) Brain Bank Criteria; classified as Hoehn and Yahr Stage II-IV in “on” state; had suffered from motor fluctuations associated with fluctuating idiopathic PD and a minimum of a 2-hour average daily “off” time; and showed dopaminergic responsiveness as defined by ≦30% change (reduction) in unified Parkinson's disease rating scale (UPDRS) III score compared to the pre-dose value. Patients had to be optimised on oral therapy, including levodopa (LD) not greater than 1500 mg/day (in combination with decarboxylase inhibitors) at least 30 days before screening; also, patients should have been receiving (for at least 30 days), or have received in the past, but discontinued due to adverse effects (AEs), at least 1 of the following types of medications: dopamine agonist (DA), catechol-O-methyltransferase inhibitor (COMT), or monoamine oxidase B inhibitor (MAOB).

### In-Clinic Dosing Titration Period

Patients were reminded to take domperidone (or equivalent antiemetic) for the 3 days before each In-Clinic Dosing Titration Period visit and instructed to minimise alcohol intake and to not eat food after midnight prior to each of these visits (sustenance permitted at investigator's discretion). Also, patients were instructed to withhold doses of LD and DA treatment (and any other anti-PD medication) after midnight prior to these visits.

Before the first dose of study treatment was administered at each visit, UPDRS III, a disease state assessment, FVC/FEV1, and safety assessments (including vital signs with orthostatic challenge, ECG recordings, laboratory safety tests, and AE/concomitant medication review) were conducted while patients were in an “off” state. Also, patients were trained in the use of the Aspirair® inhaler (with empty blisters).

After administration of the study dose, the patient confirmed conversion to an “on” state and the investigator recorded the UPDRS III (at 10, 20, and 40 minutes post-dose), as well as safety and lung function assessments, conducted post-dose.

Up to 2 doses of the same strength of study drug were administered during each visit. Where the first dose was tolerated and efficacious, a second dose at the same strength was administered after the 40-minute post-dose assessments were completed. After the second dose, post-dose assessment of adverse events was conducted. Where this second dose was also tolerated, the patient proceeded to the At-Home Dosing Period; if it was not tolerated, the patient was withdrawn from the study and was asked to return to the clinic after about 1 week for the Close-Out Visit. Where the first dose was tolerated but was not efficacious, the patient proceeded to the next visit on another day for further dose titration. Where the first dose was not tolerated, the patient was withdrawn from the study and asked to return to the clinic after about 1 week for the Close-Out Visit.

Patients were instructed to volunteer when they converted to an “on” state. Where the patient did not convert to the “on” state 40 minutes after dosing with study drug and where the patient experienced a particularly uncomfortable “off” episode, he or she could administer appropriate usual PD medication. All planned and outstanding post-dose safety assessments were conducted prior to administration of this PD medication. The patient was not given any further study drug at the current visit, and the patient was asked to return to the clinic after 1 to 14 days for the next titration visit.

Visit 1: Patients were randomised to study treatment, and this visit occurred 3 to 14 days after the Screening procedures were completed. The In-Clinic Dosing Titration Period procedures described above were also performed. The first dose of study treatment (1.8 mg delivered dose of apomorphine inhalation powder or placebo) was to be self-administered by patients (or through carer input) under nurse/doctor supervision.
Visit 2: Visit 2 occurred 1 to 14 days after Visit 1. The same In-Clinic Dosing Titration Period procedures occurred, but the study medication given was 2.8 mg delivered dose of apomorphine inhalation powder or placebo.
Visit 3: Visit 3 occurred 1 to 14 days after Visit 2. The same In-Clinic Dosing Titration Period procedures occurred, but the study medication given was 4.0 mg delivered dose of apomorphine inhalation powder or placebo.
Visit 4: Visit 4 occurred 1 to 14 days after Visit 3. The same In-Clinic Dosing Titration Period procedures occurred, but the study medication given was 5.8 mg delivered dose of apomorphine inhalation powder or placebo.

Patients who achieved an efficacious and tolerable dose of study medication at any visit (their optimal dosing visit) during the In-Clinic Dosing Titration Period of the study were allowed to proceed to the At-Home Dosing Period on this efficacious and tolerable dose. In addition, patients who tolerated the top dose at Visit 4, even if it was not efficacious, proceeded to the At-Home Dosing Period on this tolerable dose.

### At-Home Dosing Period

The at-home dosing period (up to 32 days) required patients to take their study medication for the treatment of sudden “on-off” or “wearing-off” episodes up to 5 times per day i.e. up to 5 times in 24 hours. Patients were asked to wait at least 25 minutes after taking the study treatment before taking any alternative usual PD medication, if needed. Patients were instructed to minimise their alcohol intake and to take domperidone as an anti-emetic throughout the At-Home Dosing Period. Also, patients were told to call the clinic if they experienced any intolerable adverse events during this period.

Patients continued to record their usual (non study) PD medication in the Diary Card during the last 3 consecutive days prior to Visit 5 and to Visit 6, plus they were asked to add the following information each day throughout the At-Home Dosing Period: the date and time of study medication inhalation, whether the dose worked, and—if the dose worked—the time it started to work and the time it stopped working, and if the dose was taken to treat a sudden “on-off” or a “wearing-off” episode. Patients were also instructed to complete the following information on the Diary Cards during the last 3 consecutive days prior to Visit 5 and prior to Visit 6: the date, time asleep, time “off”, time “on” without dyskinesias, time “on” with non-troublesome dyskinesias, and time “on” with troublesome dyskinesias.

Visit 5: About halfway through the At-Home Dosing Period (ie, 14±2 days after the last In-Clinic Dosing Titration Period visit), patients returned to the clinic for Visit 5. No anti-PD treatment was administered after midnight prior to this visit, and patients were asked to fast from midnight until the post-dose assessments were completed (if applicable). Safety laboratory tests were repeated. The investigator examined the Diary Card information to assess adequate efficacy and tolerability with the current dose, and confirmed appropriate completion of the Diary Card and use of the Aspirair® inhaler. Safety assessments were also conducted; if an escalated dose is administered to the patient, efficacy assessments were performed as well.

If adequate efficacy and tolerability of the current at-home dose of study medication was confirmed, the patient continued with the applicable Visit 5 safety assessments and resumed the At-Home Dosing Period at his or her current dose level. In the following scenarios of increasing and decreasing dose levels, if the efficacy was adequate, but the tolerability was questionable, the investigator first discussed the patient status with the patient before deciding on withdrawal from the study.

If the current at-home dosing regimen was not adequate, the patient was given the next higher dose during the clinic visit, and the patient continued with the applicable post-dose Visit 5 safety and efficacy assessments. Where this escalated dose was tolerated and efficacious, the patient was given a second dose; if this second dose was tolerated, the patient resumed the At-Home Dosing Period at this escalated dose. Where the escalated dose (first or second dose) was not tolerated, he or she was withdrawn from the study and asked to return to the clinic after about 1 week for the Close-Out Visit. Where the escalated dose was tolerated, but not efficacious, the patient was given the opportunity to try an increased dose level either at this visit or at an additional visit within 1 week. Where the current at-home dosing regimen was not adequate and the patient was already at the highest dose, he or she was withdrawn and asked to return to the clinic after about 1 week for the Close-Out Visit.

Where the current at-home dose was not tolerated, the dose of study medication was reduced to the next lowest dose, and the patient continued with the applicable Visit 5 safety procedures and assessments. Where this lower dose was tolerated and efficacious, the patient was given a second dose; if this second dose was tolerated, the patient resumed the At-Home Dosing Period at this decreased dose. Where the lower dose was not tolerated and/or not efficacious during the clinic visit, he or she was withdrawn and asked to return to the clinic after about 1 week for the Close-Out Visit. If the current at-home dose was not tolerated and the patient was already at the lowest dose, he or she was withdrawn and asked to return to the clinic after about 1 week for the Close-Out Visit.

Visit 6 (End-of-Treatment Visit): Visit 6 occurred 14±2 days after Visit 5. At this End-of-Treatment Visit, Diary Cards were collected, drug compliance checked, and the following procedures performed: ECG recordings, FVC/FEV1, vital signs (without orthostatic challenge), clinical laboratory tests, and assessment of AEs and concomitant medications.

Visit 7 (Close-Out Visit): A Close-Out Visit occurred up to 7 days after Visit 6 or when a patient discontinued early from the study. Procedures included a physical examination, FVC/FEV1, vital signs (with orthostatic challenge), ECG recordings, and assessment of AEs and concomitant medications; clinical laboratory tests were done at this visit if the patient discontinued early, or if the patient had clinically significant results at the End-of-Treatment Visit, those laboratory tests were repeated at the Close-Out Visit. An exit pregnancy test was carried out in appropriate females.

The study endpoint evaluation included the determination of the maximum change in total UPDRS III score from pre-dose to post-dose during the in-clinic dosing titration period, the change in mean daily “off” time per day compared with the baseline value and the number of adverse events experienced by patients assessed.

### Referenced Clinical Studies

Results from other clinical studies have been compared to the current VR040/2/008 study. A recently completed Phase IIa clinical study, VR040/2/003, which evaluated the safety, efficacy and pharmacokinetics of apomorphine inhalation formulation (in-clinic) resulted in statistically significant UPDRS III improvements in the active group compared to the placebo group.

APO202 (R. B. Dewey et al; 2001) assessed the safety and efficacy of subcutaneous apomorphine hydrochloride administration for “off” state episodes in patients with PD at both in-clinic and at-home settings.

The purpose of the APO302 study (R. F. Pfeiffer et al; 2006) was to review the efficacy of intermittent subcutaneous apomorphine as an acute therapy for “off” episodes in patients with advanced PD who had received treatment for months.

The melevodopa/carbidopa study programme (F. Stocchi et al; 2010), compared the effectiveness of oral melevodopa/carbidopa effervescent tablets with standard oral levodopa/carbidopa tablets. The study revealed that the melevodopa/carbidopa effervescent oral tablets were more rapid and provided consistent absorption resulting in quicker and a more predictable therapeutic response to standard levodopa/carbidopa oral tablets.

S90049 is a novel sublingual formulation of non-ergoline D2-D3 agonist piribedil (0. Rascol et al; 2010). This study assessed the efficacy and safety of S90049 in aborting “off” episodes of PD to subcutaneous apomorphine.

### EXAMPLES

### Example 1

### Demographics

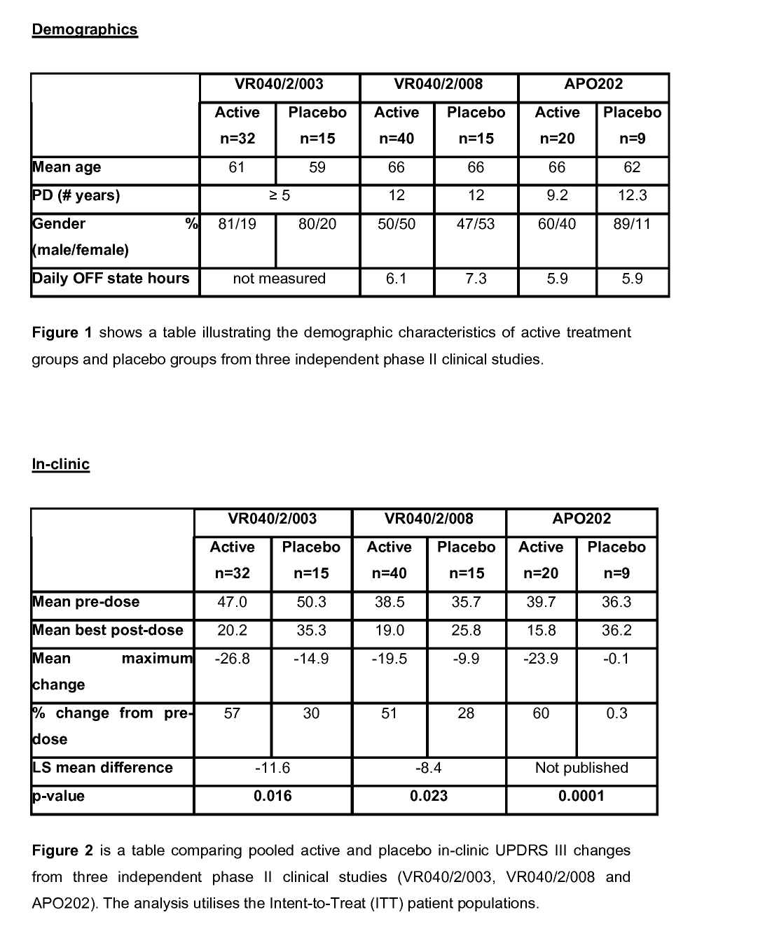

Demographic characteristics including mean age, length of time diagnosed with PD, gender and daily period in “off” state were compared in three independent phase II clinical studies (VR040/2/003, VR040/2/008 and APO202). Studies were seen to be comparable in terms of each of the demographic characteristics recorded except for daily period in “off” state, which was not measured in the VR040/2/003 study. FIG. 1 shows a table illustrating the demographic characteristics of active treatment groups and placebo groups from three independent phase II clinical studies. The active study treatment group and the placebo group were comparable for the VR040/2/008 study.

### Example 2

### Efficacy in-Clinic

One of the co-primary efficacy end points was the maximum change in total UPDRS III score from pre-dose to post-dose during the in-clinic dosing titration period. FIG. 2 summarises active and placebo in-clinic UPDRS III changes for the ITT populations from three independent phase II clinical studies (VR040/2/003, VR040/2/008 and APO202). The active treatment group from the VR040/2/008 study displayed a clinically relevant and statistically significant improvement, compared with the placebo group (p=0.023).

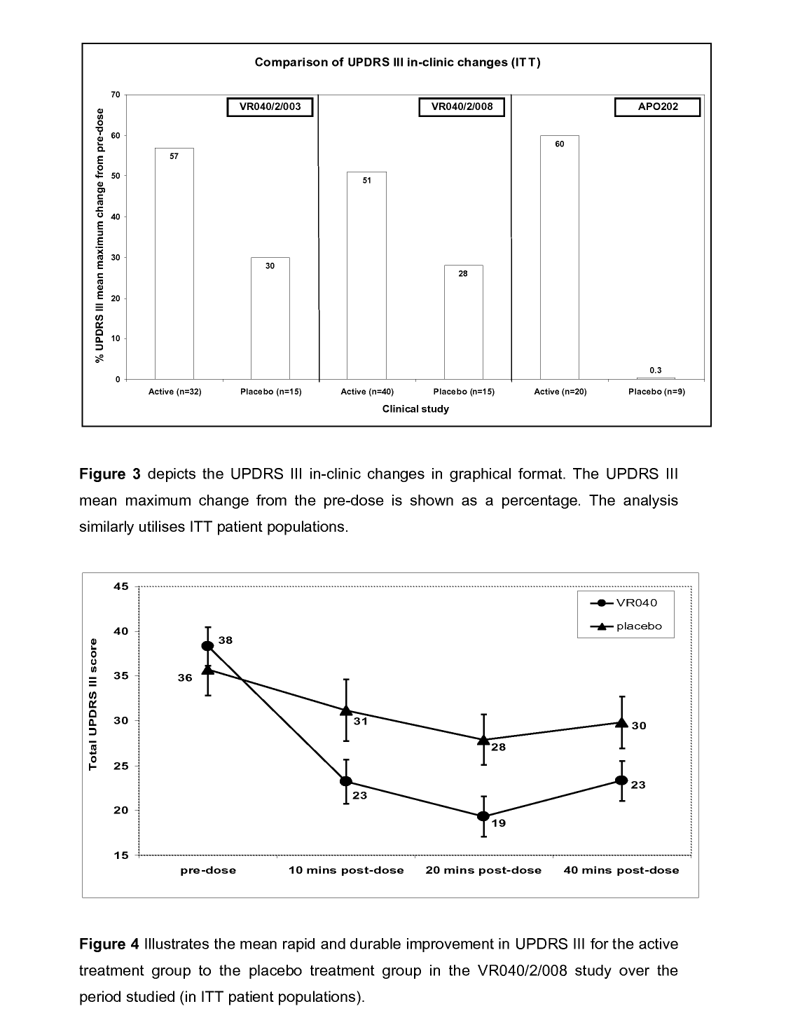

The UPDRS III in-clinic mean maximum changes from the pre-dose as a percentage in three independent clinical studies is summarised in FIG. 3. The VR040/2/008 active treatment group demonstrated a 51% UPDRS III mean maximum change from the pre-dose compared to a 28% change seen in the placebo group (ITT patient populations).

FIG. 4 illustrates the mean rapid and durable improvement in UPDRS III for the active treatment group which is superior to the placebo treatment group in the VR040/2/008 study over the period studied (ITT patient populations). UPDRS III assessment was conducted pre-dose and at 10, 20 and 40 minute intervals post-dose, regardless of time of conversion. This improvement in UPDRS III at 10 minutes closely correlates with the patient reported median onset of therapeutic benefit at 5.5 minutes post inhalation of active treatment.

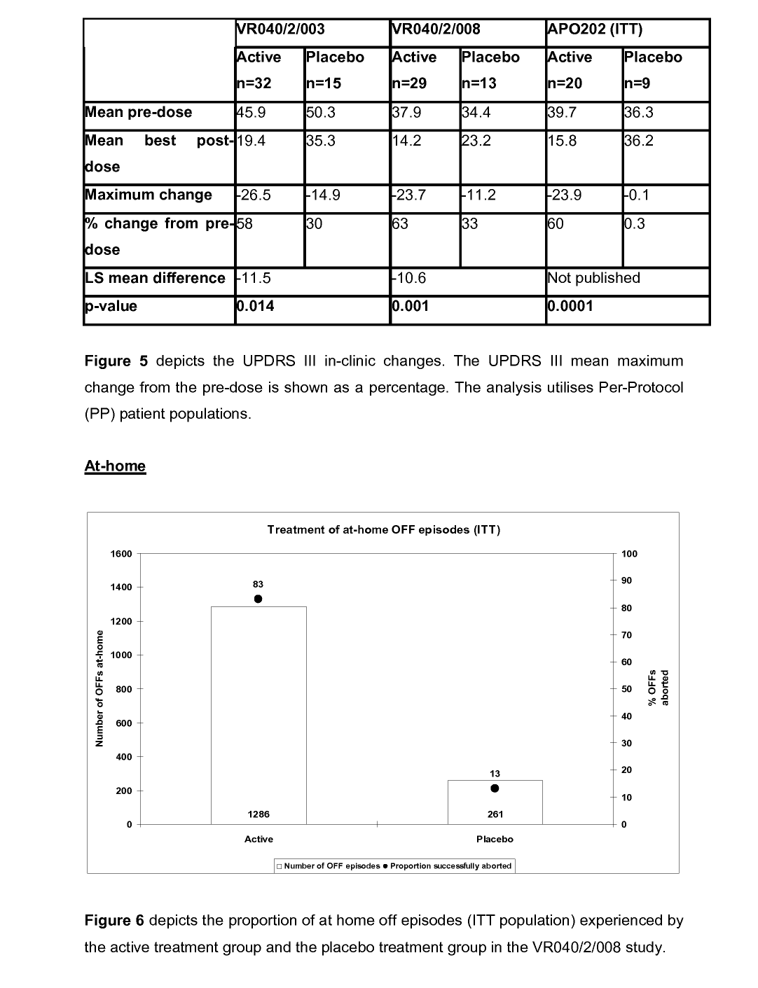

FIG. 5 compares the active and placebo in-clinic UPDRS III changes from three independent phase II clinical studies (VR040/2/003, VR040/2/008 and APO202). The analysis utilises Per-Protocol (PP) patient populations for VR040/2/003 and VR040/2/008 comparisons and ITT patient populations for the APO202 study. The VR040/2/008 active treatment group demonstrated a 63% change from the pre-dose compared to a 33% change observed with the placebo group.

### Example 3

### Efficacy at-Home

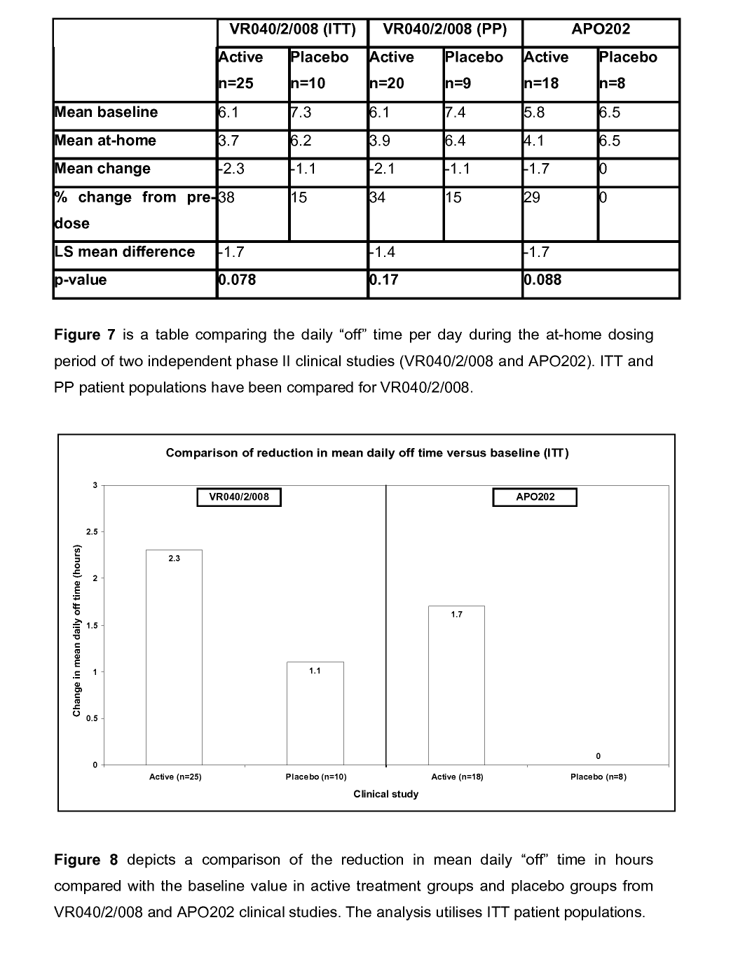

Another of the co-primary efficacy end points was the change in “off” time per day compared with the baseline value. FIG. 6 illustrates the increased ability of active treatment to reproducibly convert patients from “off” to the “on” state with 83% and 13% of active (n=1286) and placebo (n=261) treated OFF episodes being successfully aborted. FIG. 7 compares active and placebo changes in the daily “off” time per day during the at-home dosing period of 2 independent phase II clinical studies (VR040/2/008 and APO202). The active treatment group from the VR040/2/008 study was shown to reduce the time patients were in an “off” state by over 2 hours, a change considered by investigators to be highly clinically relevant, when compared with the placebo group. The change in mean daily “off” time in hours has also been depicted graphically in FIG. 8, which specifically compares the reduction in the mean daily “off” time in the active treatment and placebo groups from the VR040/2/008 and APO202 studies.

Secondary efficacy endpoints measured included; mean time to therapeutic benefit; mean daily period asleep; mean daily duration of “on” state without dyskinesias; mean daily duration of “on” state with non-troublesome dyskinesias and mean daily duration of “on” state with troublesome dyskinesias.

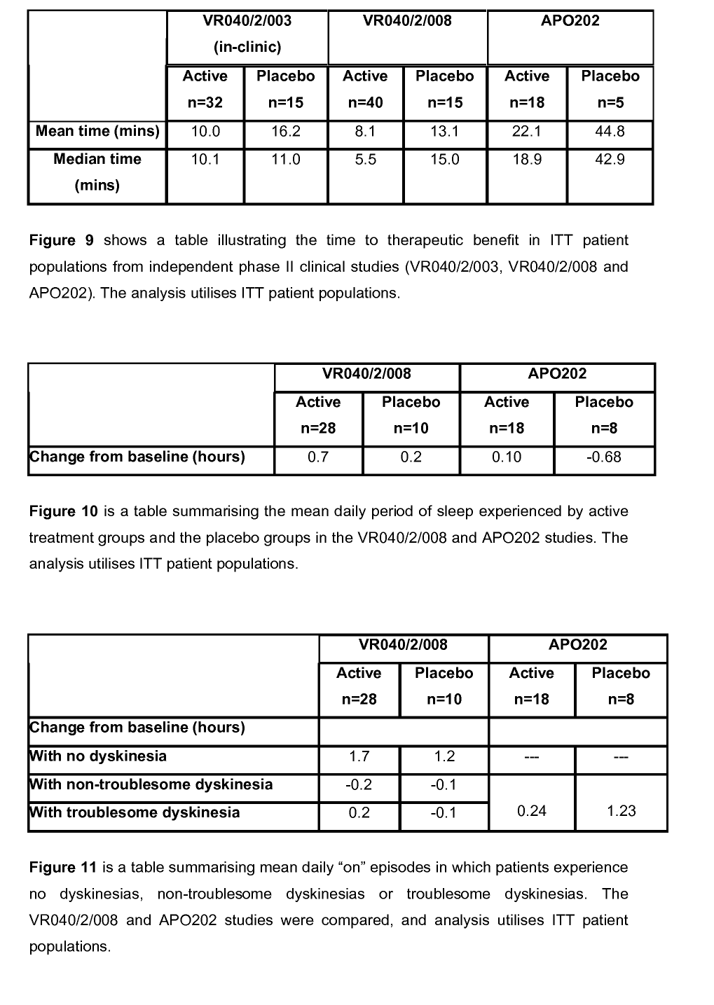

The mean time to therapeutic benefit was recorded in-clinic period of the VR040/2/003 study and at-home dosing period of the VR040/2/008 study (ITT patent populations were analysed). The mean time to therapeutic benefit observed in the treatment group of the in-clinic period was 10 minutes (placebo 16.2 minutes), which was marginally slower than the mean time observed in the treatment group of the at-home period, which was 8.1 minutes (placebo 13.1 minutes). In the APO202 study, the active treatment group was shown to take a mean time of 22.2 minutes to reach therapeutic effect (see FIG. 9).

The mean daily period of sleep experienced by active treatment groups and placebo groups in the VR040/2/008 study were compared against the APO202 study, see FIG. 10 (ITT patient populations were analysed). The change in the mean daily period asleep from the baseline reported by the VR040/2/008 active treatment group was 0.7 hours compared to 0.2 hours in the placebo group. Although the duration of sleep experienced by the treatment group was longer, a similar number of patients in both groups reported an increase in time asleep (58% for the active treatment group compared to 60% for placebo). In the APO202 study, the active treatment group was only shown to have a 0.10 hour change in the mean daily period asleep from the baseline.

On 83 occasions during the at-home period active treatment was administered between midnight and 06:00 am to treat “off” episodes. On 64% of occasions such “off” episodes were successfully aborted. In addition, on 68% of occasions patients did not need to administer a second active dose within a 4 hour interval indicating subjects being able to return to sleep for at least a 4 hour period and the ability of treatment to address “night-time off” episodes.

The mean daily “on” time in which VR040/2/008 active treatment groups and placebo groups experienced no dyskinesia, non-troublesome dyskinesia or troublesome dyskinesia was examined. Patients were asked to record on diary cards their predominant state (asleep, off, on no dyskinesia, on non-troublesome dyskinesia, on troublesome dyskinesia) in half-hour periods for 3 days prior to clinic visit the output of which is summarised in FIG. 11. This resulted in a 1.7 hour and 1.2 hour increase in the mean daily “on” associated with no dyskinesia compared to baseline for active and placebo groups respectively. Dyskinesia was not reported as an adverse event by any patient during the VR040/2/008 in-clinic or at-home phase. The mean daily duration of “on” state with non-troublesome dyskinesia and troublesome dyskinesias was 0.24 hours and 1.23 hours in the active and placebo groups of the APO202 study.

There is no evidence to suggest that active treatment resulted in an increased incidence of dyskinesia (troublesome of non-troublesome) compared to baseline. Furthermore, there is no evidence to suggest of increased dyskinesia incidence or severity for those patients increasing their at-home study dose at Visit 5. Such an outcome is different to that reported during the clinical evaluation of subcutaneous apomorphine. During four pivotal clinical studies (APO202, APO301, APO302 and APO303) dyskinesia was reported at a higher incidence. More specifically, during study APO202 15 of 20 patients (75%) reported an increased dyskinesia severity compared to baseline with 11/16 (69%) reporting incidences of dyskinesia during study APO301.

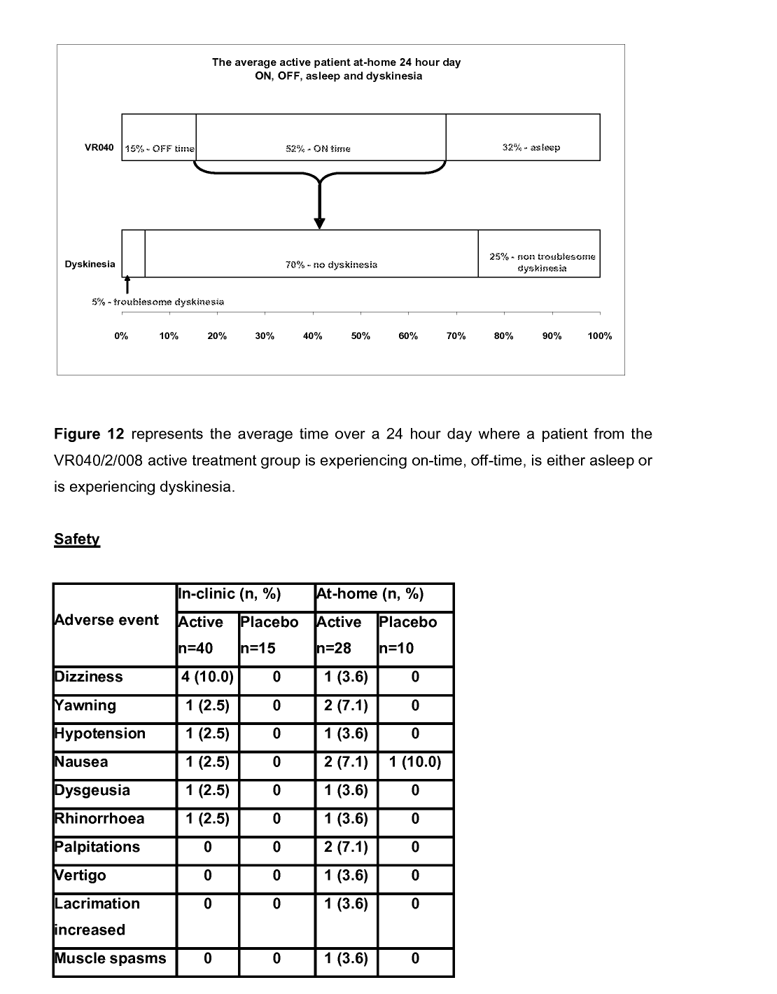

FIG. 12 represents the average time over a 24 hour period where a patient from the VR040/2/008 active treatment group is experiencing “on” time, “off” time or is either asleep or is experiencing dyskinesia. Most of the active patient daily “on” time was dyskinesia free (70%) with the remainder being associated with non troublesome (25%) and troublesome dyskinesia (5%).

### Example 4

### Safety

### Adverse Events

Treatment-related adverse events (AEs) during the in-clinic and at-home phases were investigated. An adverse event is any untoward medical occurrence or un-desired “side-effect” that occurs in a patient as a result of the administered medical treatment. No untoward safety concerns were identified in the current VR040/2/008 study or in the previously completed phase IIa trials, VR040/001 and VR040/2/003.

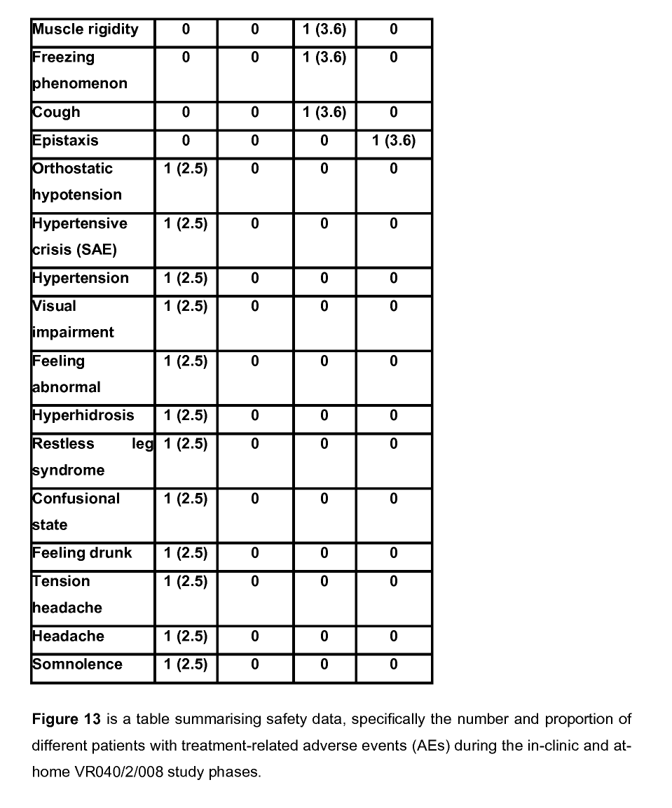

The number and proportion of different patients with treatment-related AEs during the in-clinic and at-home VR040/2/008 study phases have been summarised in FIG. 13.

The in-Clinic Phase:

The in-clinic phase did not give rise to any serious or severe treatment-related AEs, and only 3 patients withdrew from the study due to experiencing an AE. Of the 40 patients randomised to active treatment, 10 reported a total of 23 treatment-related AEs, of which 17 were mild in severity and 6 moderate. No placebo randomised patients reported any treatment-related AEs. Furthermore no patients spontaneously reported dyskinesia as an AE during the in-clinic phase.

The at-Home Phase:

There were similarly no reports of any serious or severe treatment-related AEs, and only 2 patients withdrew from the study. Of the 28 patients randomised to active treatment, 6 experienced a total of 18 treatment-related AEs of mild or moderate severity. 2 placebo randomised patients reported 2 mild treatment-related AEs. Furthermore no patients spontaneously reported dyskinesia as an AE during the at-home phase.

The approved dosing interval for the successive administration of subcutaneous apomorphine doses is 2 hours. During study VR040/2/008 a number of patients administered successive inhaled apomorphine doses within 1 and 2 hours. This reduced dosing interval was not associated with an increased adverse event incidence thereby further exemplifying the improved safety of the inhaled delivery route.

It should also be noted that some patients, despite protocol instructions, did not administer concomitant anti-emetic treatment. Despite this, there was no increased incidence of adverse events such as nausea and vomiting. This outcome supports the potential for reduced concomitant anti-emetic use with inhaled apomorphine.

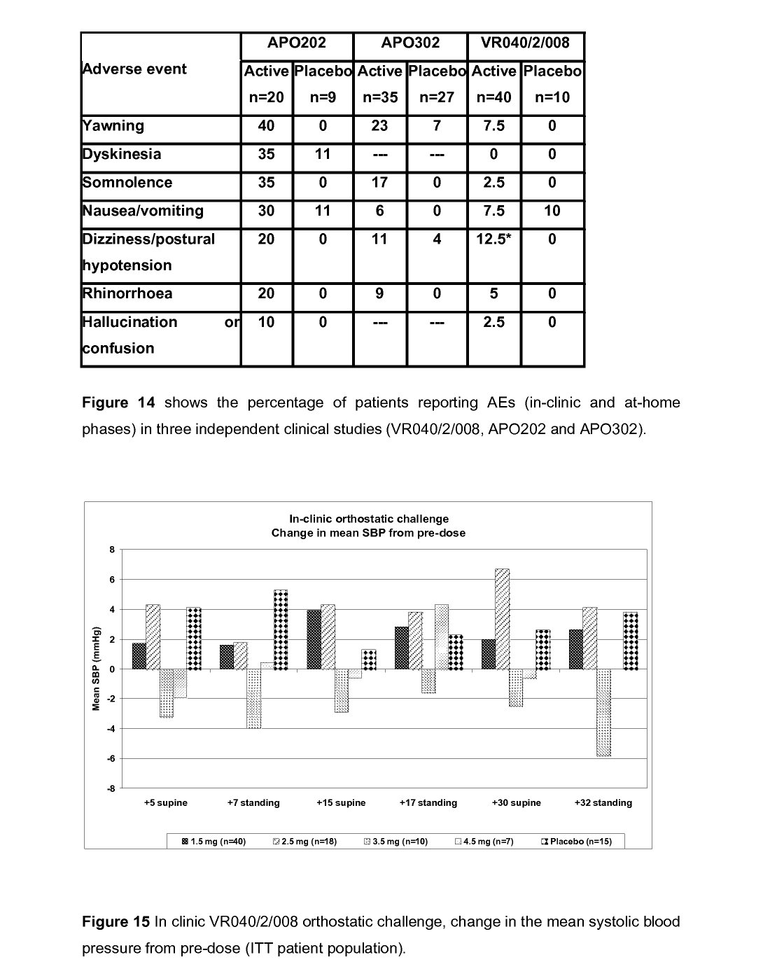

VR040/2/008 safety data was compared to subcutaneously injected apomorphine APO202 and APO302 studies. The percentage of patients reporting AEs including yawning, somnolence and Rhinorrhoea (during in-clinic and at-home dosing periods) was noticeably lower in patents treated with the inhaled apomorphine formulation. There were no reports of dyskinesia in the VR040/2/008 study compared to the APO202 study in which 35% of patients from the active group and 11% from the placebo group reported signs of dyskinesia, as seen in FIG. 14. 1 patient from the VR040/2/008 active treatment group did report dizziness and/or postural hypotension which resulted in a percentage (12.5%) that was lower or near-comparable to the APO202 and APO302 studies respectively.

### Vital Signs

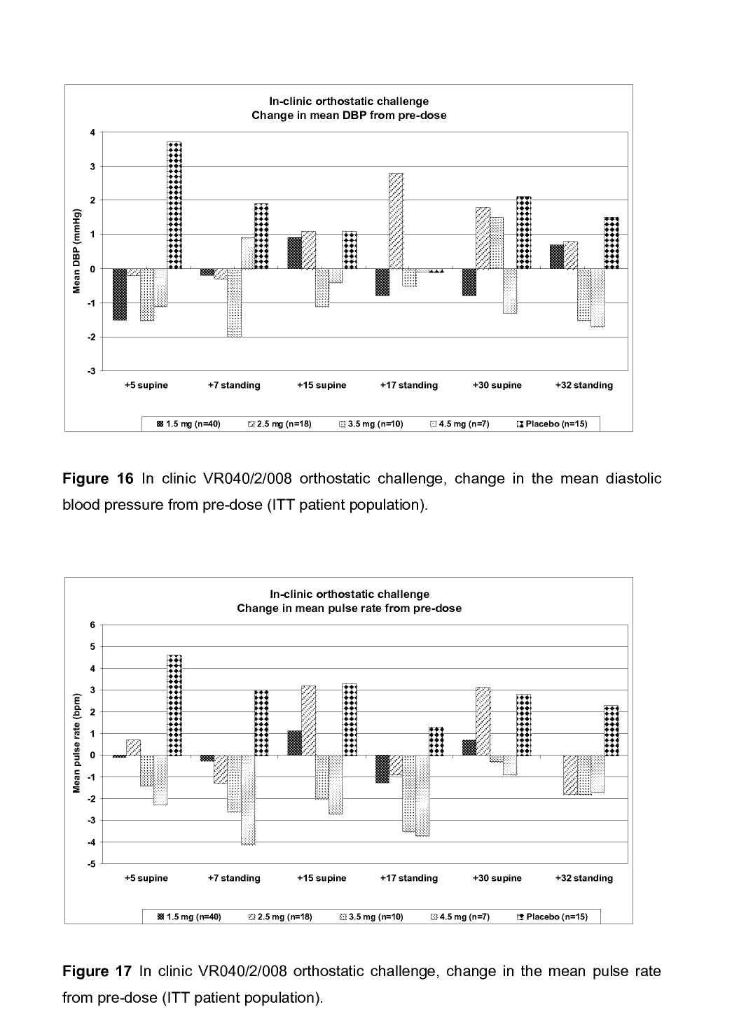

The phase II VR040/2/008 clinical study also assessed participating patient's vital signs, specifically blood pressure and pulse rates. Vital signs were assessed at Screening, for each treatment administration at pre-dose as well as 5, 15, and 30 minutes post-dose at Visits 1 through 5, and also at the End-of-Treatment Visit and the Close-Out Visit. For all the time points except for Visit 6 and, providing there is no dose change, Visit 5, measurements were recorded after the patient had been in the supine position for 5 minutes and then after the patient had been standing for 2 minutes (i.e. the orthostatic challenge). FIG. 15 illustrates the in clinic change in the mean systolic blood pressure from pre-dose (ITT patient population). FIG. 16 illustrates the in clinic change in the mean diastolic blood pressure from pre-dose (ITT patient population). FIG. 17 illustrates the in clinic change in the mean pulse rate from pre-dose (ITT patient population).

All the vital sign mean changes observed were of relatively small magnitude, generally +/−10%. For systolic blood pressure the mean changes were less than 8 mm Hg. For diastolic blood pressure the mean changes were less than 4 mm Hg and for heart rate the mean changes were less than 5 bpm.

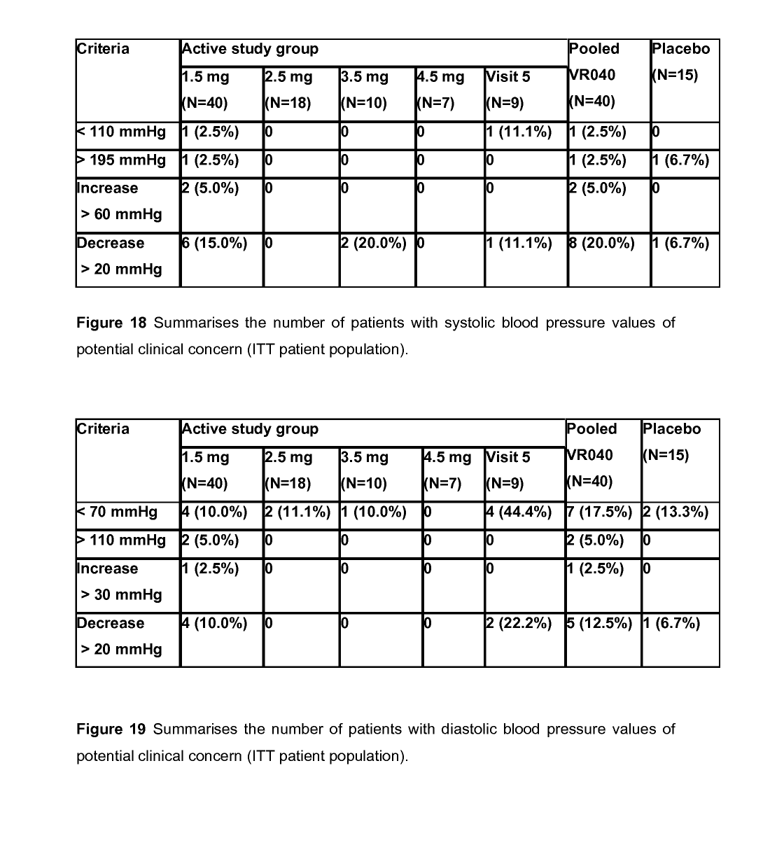

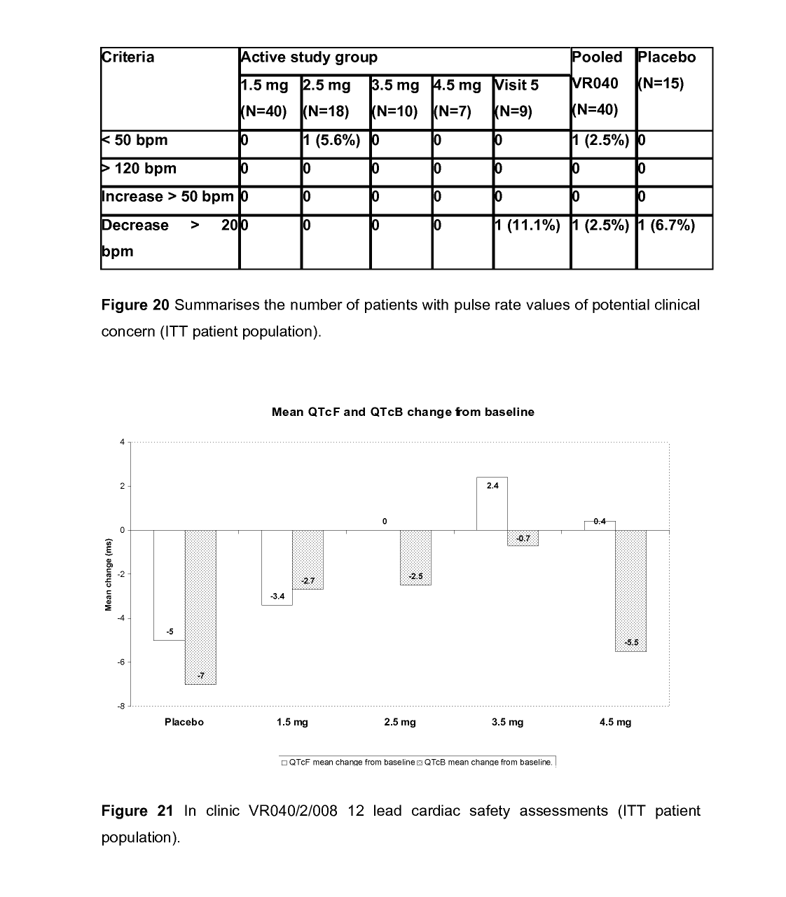

The study also examined the number/proportion of patients with systolic blood pressure values (FIG. 18), diastolic blood pressure values (FIG. 19) and pulse rate values (FIG. 20) that were of potential clinical concern. Although some vital sign values did meet the pre-defined criteria for clinical concern for the VR040 programme, very few were also noted by investigators as clinically significant and the majority of patients continued to the at-home phase of the study.

Despite every patient being subjected to a strenuous orthostatic challenge the reductions in vital signs were of small magnitude and correlating to the excellent adverse event profile and low incidence of typical dopaminergic stimulation responses such as hypotension.

### ECG Assessments

A further safety aspect examined was cardiac safety. Electrocardiogram (ECG) measurements were taken using a twelve lead continuous Holter ECG and traditional twelve lead methodology. Three consecutive and separate twelve lead ECG assessments were performed on patients that were relaxed and in a sitting position at Screening, at pre-dose Visits 1 through 5 (and also a single measurement at 40 minutes post-dose), at the End-of-Treatment Visit and at the Close-Out Visit. Measurements were also taken at the following fixed time points: 2; 9; 25 and 35 minutes post-dose. FIG. 21 shows the mean QTcF and QTcB changes from the baseline for the VR040/2/008 placebo treatment group and for each active treatment administration group.

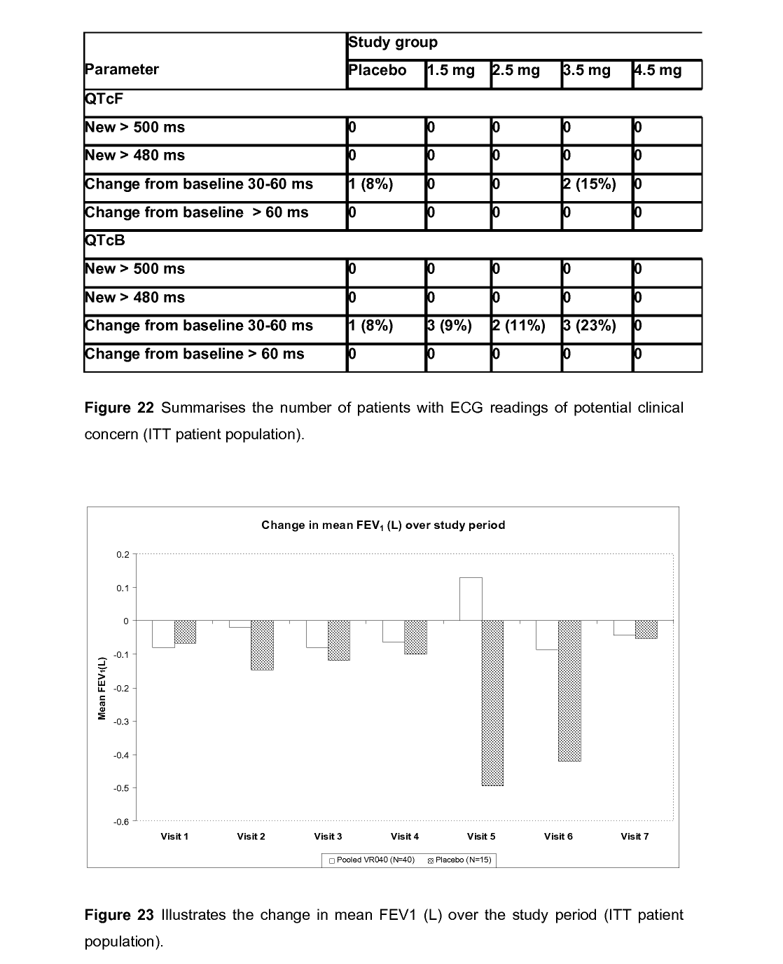

The number/portion of patients with ECG readings of potential clinical concern (ITT patient population) was similarly examined and the results are shown in FIG. 22.

No patient reported QTcB or QTcF changes (relative to baseline) or absolute values of clinical concern. This provides further evidence of excellent safety with inhaled apomprphine.

### Lung Function

Lung function assessments were also performed and were conducted in accordance with current American Thoracic Society (ATS) guidelines using a spirometer. FVC/FEV₁ readings were taken at Screening, at pre-dose and about 40 minutes post-dose at Visits 1 through 5 for each treatment administration, and at the End-of-Treatment Visit and the Close-Out Visit. Patients with FEV₁ results 65% predicted at Screening were excluded from the study. Predicted FEV1 values were determined using the European Community for Coal and Steel Guidelines for Standardised testing. FIG. 23 depicts the pre-dose change from baseline (screening) in mean FEV₁ (L) over the VR040/2/008 study period (ITT patient population).

There was no evidence of any causal relationship between treatment administration and lung function.

### Example 5

### Comparisons with Other Development Programmes

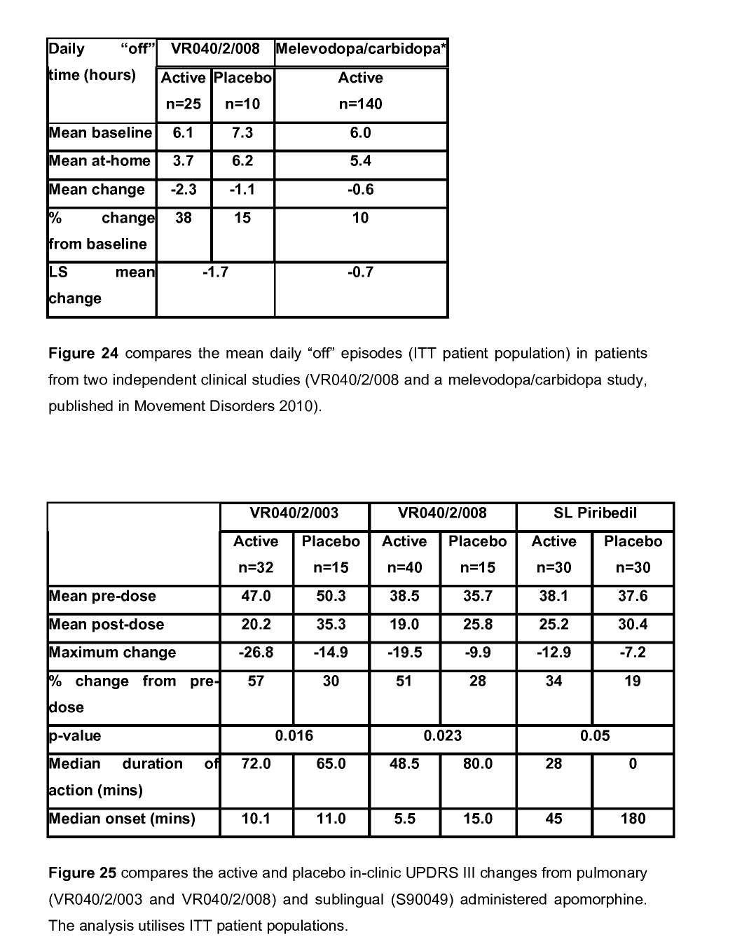

The mean reduction in daily “off” time (ITT patient population) experienced by the active treatment group and placebo group in the VR040/2/008 study were compared against a 12 week at-home melevodopa/carbidopa study (F. Stocchi et al; 2010). Melevodopa hydrochloride with carbidopa in effervescent tablets is a readily soluble PD oral tablet formulation. FIG. 24 demonstrates that the percentage change from the baseline in the VR040/2/008 active group was 38% (15% in placebo) compared to a 10% change observed in the Melevodopa/carbidopa active group.

FIG. 25 compares the active and placebo in-clinic UPDRS III changes from pulmonary (VR040/2/003 and VR040/2/008) and sublingual (S90049) administered apomorphine. The analysis utilises ITT patient populations. The median duration of therapeutic effect observed for the active VR040/2/008 treatment group was 48.5 minutes for the treatment of “wearing off” episodes, 59.9 minutes for the treatment of “sudden off” episodes and 56.5 minutes for the treatment of all “off” episodes.

### Example 6

### Pharmacokinetic Profile of Apomorphine by Inhalation

A recently completed Phase II clinical study, VR040/2/003, evaluated the safety, efficacy and pharmacokinetics of apomorphine inhalation formulation (in-clinic) at approximate nominal doses of 3200 μg, 4800 μg, 6400 μg and 9000 μg (equating to approximate fine particle doses of 1500 μg, 2300 μg, 3000 μg and 4000 μg, respectively).

Blood samples for pharmacokinetic analysis were taken pre-dose and at the following intervals post-dose administration: 1 minute, 4 minutes, 7 minutes, 20 minutes, 30 minutes, 50 minutes, 70 minutes and 90 minutes.

The following pharmacokinetic parameters were calculated: area under the concentration-time curve between 0 and 90 minutes (AUC₀₋₉₀), area under the concentration-time curve between 0 minutes and infinity (AUC_{0-inf}), time to maximum plasma concentration (tₘₐₓ), maximum drug concentration in plasma (Cₘₐₓ), terminal half life (t_{1/2}) and terminal rate constant (λz).

The results are summarised in Table 1 and refer to the mean values obtained from the patient populations tested for each of the above-mentioned doses. Pharmacokinetic analysis confirmed that a very rapid attainment of mean tₘₐₓ of 2 to 7.3 minutes after dose administration was observed.

TABLE 1

| | | Treatment Group | | | |
|---|---|---|---|---|---|
| | | VR040 | VR040 | VR040 | VR040 |
| | | 3200 μg | 4800 μg | 6400 μg | 9000 μg |
| | | (1500 μg) | (2300 μg) | (3000 μg) | (4000 μg) |
| Parameter | Statistic | (N = 5) | (N = 1) | (N = 3) | (N = 1) |
| AUC₍₀₋₉₀₎ | Mean (SD) | 103.89 (71.58) | 642.35 | 385.62 (190.10) | 645.52 |
| (ng · min/mL) | | | | | |
| AUC_{(0-inf)} | Mean (SD) | 171.58 (145.87) | 675.77 | 458.17 (203.63) | 817.17 |
| (ng · min/mL | | | | | |
| Cₘₐₓ (ng/ml) | Mean (SD) | 3.68 (2.55) | 26.60 | 16.00 (15.47) | 21.80 |
| λ_{z} (l/min) | Mean (SD) | 0.018 (0.008) | 0.03 | 0.02 (0.00) | 0.02 |
| tₘₐₓ (min) | Mean (SD) | 2.2 (1.6) | 7.0 | 7.3 (11) | 4.0 |
| t_{1/2} (min) | Mean (SD) | 58.73 (31.53) | 20.32 | 32.44 (3.51) | 31.23 |

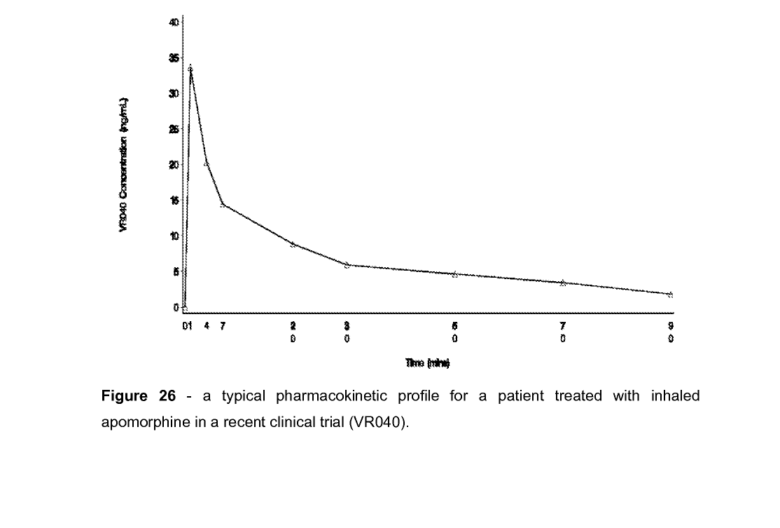

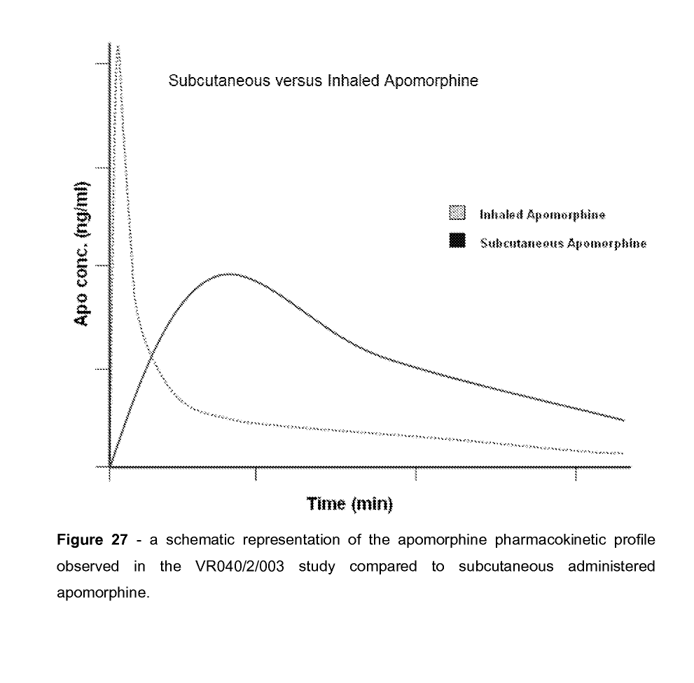

FIG. 26 is a typical individual patient profile of apomorphine plasma concentration versus time post oral inhalation. This profile is representative of the results obtained in the study and demonstrates the very distinctive pharmacokinetic profile that was observed. The profile illustrates rapid systemic absorption, with maximum apomorphine plasma concentrations observed within minutes of dose administration (in this case, about 2 minutes).

While not wishing to be bound by theory, it is believed that the achieved maximum plasma exposure (Cₘₐₓ) may be sufficient to induce a therapeutic response in the Parkinson's patient i.e. conversion from the OFF to the ON state. Minutes after achieving Cₘₐₓ, the apomorphine plasma concentration declines rapidly. Consequently, the period that the apomorphine plasma concentration remains high is short and is considered to be of insufficient duration to induce the adverse events typically associated with dopaminergic stimulation. It is believed that this observation is validated by comparing safety data from the more recent VR040/2/008 study and previous subcutaneous data. FIG. 27 is a schematic representation of the apomorphine pharmacokinetic profile observed in the VR040/2/003 study compared to subcutaneous administered apomorphine. Conventional thinking dictates that patients exposed to high apomorphine Cₘₐₓ concentrations have a greater probability of experiencing troublesome side effects typically associated with dopaminergic treatment e.g. nausea, dizziness, and somnolence. It would therefore be expected that subjects receiving inhaled apomorphine would report a greater incidence and severity of adverse events compared to those administered subcutaneous apomorphine which is associated with a lower Cₘₐₓ value (see FIG. 26). However, studies VR040/2/003 and VR040/2/008 involving 102 PD patients indicate the opposite is actually the case, with inhaled apomorphine PD subjects reporting a significantly lower incidence of side effects. This observation illustrates the importance of a rapid apomorphine plasma concentration decline within minutes of Cₘₐₓ. The distinctive profile seen in VR040/2/003 is expected to be replicable and is due to a number of influential factors:

(A) Route of administration—administration of the apomorphine formulation by oral inhalation (e.g. oral pulmonary inhalation) appears to provide increased delivery efficiency, increased bioavailability and consistent absorption and appears to deliver an ultimately faster and more predictable clinical effect whilst avoiding the side effects associated with other routes of administration;
(B) Formulation—the dry powder formulations described herein are both chemically and physically stable allowing for the consistent targeted delivery of apomorphine to the pulmonary system. The formulations may be formulated with or without additive material and/or alternatively with or without one or more excipient materials; and
(C) Inhalation device—any inhalation device as described herein can be used. However, it appears that the dry powder formulations are most suitably used in combination with a dry powder inhaler (e.g. a passive or active device) as described herein.

Non-limiting examples of the particular combinations that may provide the desired pharmacokinetic and side-effect profile include those described below:

### (I) Combination A

(i) administration is by pulmonary inhalation;
(ii) the formulation comprises a dopamine agonist (e.g. apomorphine in combination with levodopa and/or a dopamine agonist that is not apomorphine); and
(iii) the formulation is delivered from an appropriate inhalation device as described herein;

### (II) Combination B

(i) administration is by oral pulmonary inhalation;
(ii) the formulation comprises apomorphine that is used at a nominal dose as described herein; and
(iii) the formulation is delivered preferably by a dry powder passive or active inhaler;

### (III) Combination C

(i) administration is by oral pulmonary inhalation;
(ii) the formulation comprises a dopamine agonist (e.g. apomorphine in combination with levodopa and/or a dopamine agonist that is not apomorphine);
(iii) the formulation further comprises an additive material such as an additive material as described herein and/or carrier particles made from one or more excipient material as described herein; and
(iv) the formulation is delivered from an appropriate inhalation device as described herein; and

### (IV) Combination D

(i) administration is by oral pulmonary inhalation;
(ii) the formulation comprises apomorphine that is used at a nominal dose as described herein;
(iii) the formulation further comprises an additive material, preferably magnesium stearate and/or the formulation further comprises carrier particles made from one or more excipient material as described herein; and
(iv) the formulation is delivered preferably by a dry powder passive or active inhaler.

It will be appreciated that the above-mentioned combinations can include additional components or can be used in conjunction with a subject's current therapeutic regimen. For example, the formulations can contain more than one dopamine agonist (e.g. apomorphine and levodopa) or the formulation described herein can be used in conjunction with levodopa therapy.

Thus, targeted delivery of inhaled apomorphine in the treatment of Parkinson's disease can be achieved by exploiting the formulation and device technology as described herein.

### REFERENCES

A Randomized, Double-blind, Placebo-Controlled Trial of Subcutaneously Injected Apomorphine for Parkinsonian Off-State Events; Arch Neurol 2001; 58:1385-1392 Richard B. Dewey, Jr, MD; J. Thomas Huttin, MD, PhD; Peter A. LeWitt, MD; Stewart A. Factor, Do
Continued efficacy and safety of subcutaneous apomorphine in patients with advanced Parkinson's disease; Parinsonism and Related Disorders; 2006; Ronald F Pfeiffer, Ludwig Gutmann, Keith L. Hull Jr, Peter B. Bottini, James H. Sherry, The APO302 Study Investigators.

1. A method of treating and/or preventing the symptoms of Parkinson's disease in a subject, the method comprising:
administering apomorphine in combination with levodopa and/or a dopamine agonist that is not apomorphine to treat and/or prevent the symptoms of Parkinson's disease in the subject, wherein apomorphine is administered by inhalation.

2.-9. (canceled)

10. The method of claim 1, wherein the apomorphine is provided in a separate composition to a composition comprising levodopa and/or dopamine agonist.

11. The method of claim 10, wherein the apomorphine is administered by pulmonary inhalation.

12. The method of claim 11, wherein the apomorphine is a dry powder composition.

13. The method of claim 10, wherein the composition of apomorphine comprises at least 5% of apomorphine by weight.

14. The method of claim 10, wherein the composition of apomorphine further comprises an additive material.

15. The method of claim 14, wherein the additive material in the composition of apomorphine is magnesium stearate.

16. The method of claim 10, wherein the apomorphine composition further comprises carrier particles made from one or more excipient materials.

17. The method of claim 16, wherein the excipient materials are selected from one or more of sugar alcohols, polyols, crystalline sugars, inorganic salts, organic salts, and other organic compounds.

18. The method of claim 17, wherein the excipient materials are selected from one or more of sugar alcohols, polyols, and crystalline sugars.

19. The method of claim 18, wherein the excipient materials are one or more crystalline sugars selected from mannitol, trehalose, melezitose, dextrose, or lactose.

20. The method of claim 19, wherein the excipient materials include lactose.

21. The method of claim 16, wherein the carrier particles have an average particle size between 5 to 1000 μm.

22. The method of claim 1, wherein the apomorphine provides a therapeutic effect with duration of at least 60 minutes.

23. The method of claim 1, wherein the maximum daily dose of apomorphine is less than 30 mg.

24. The method of claim 1, wherein apomorphine has a fine particle dose of between 0.5 to 4.5 mg.

25. The method of claim 24, wherein apomorphine has a fine particle dose of between 1.5 to 3 mg.

26. The method of claim 25, wherein the fine particle dose of apomorphine is higher than 1.5 mg and less than 3 mg.

27. The method of claim 1, wherein the apomorphine is administered on demand before, or at the onset of, an off episode.

28. The method of claim 1, wherein, when dosed, the apomorphine has a Cₘₐₓ that is achieved within 10 minutes of administration by inhalation.

29. The method of claim 28, wherein the Cₘₐₓ of apomorphine is dose dependent.

30. The method of claim 1, wherein the apomorphine provides a therapeutic effect within 10 minutes of administration.

31. The method of claim 1, wherein the dopamine agonist, when present, is selected from bromocriptine, pramipexole, ropinirole, or rotigotine.

32. The method of claim 1, wherein the levodopa and/or dopamine agonist is administered orally or transdermally.

33. The method of claim 1, wherein the levodopa is administered at a maximum daily dose of 1600 mg.

34. The method of claim 33, wherein the maximum daily dose of levodopa is 1500 mg.

35. The method of claim 1 further comprising:
administering other agents that treat and/or prevent the symptoms of Parkinson's disease.

36. The method of claim 10, wherein the composition of levodopa and/or a dopamine agonist further comprises other agents that treat and/or prevent the symptoms of Parkinson's disease, wherein the composition is in a single dosage form or multiple dosage forms containing one or more active ingredients.

37. The method of claim 36, wherein the other agents are selected from one or more of further dopamine agonists, mono amine oxidase B inhibitors, aromatic L-amino acid decarboxylase inhibitors, catechol-O-methyltransferase inhibitors, anticholinergics, and antimuscarinics.

38. The method of claim 37, wherein the other agents are selected from one or more of bromocriptine, pramipexole, ropinirole, rotigotine, carbidopa, benserazide, difluoromethyldopa, α-methyldopa, selegiline, rasagiline, entacapone, tolcapone, ipratropium, oxitropium, tiotropium, glycopyro late, atropine, scopolamine, tropicamide, pirenzepine, diphenhydramine, dimenhydrinate, dicyclomine, flavoxate, oxybutynin, cyclopentolate, trihexyphenidyl, benzhexyl, darifenacin, and procyclidine.

39. The method of claim 38, wherein the other agents are selected from one or more of bromocriptine, pramipexole, ropinirole, rotigotine, carbidopa, benserazide, difluoromethyldopa, α-methyldopa, selegiline, rasagiline, entacapone, and tolcapone.

40. The method of claim 1, wherein the levodopa is provided in combination with carbidopa and, optionally, entacapone.

41. The method of claim 1, wherein the levodopa and/or dopamine agonist is administered as part of a regular therapeutic dosing regimen for the treatment of Parkinson's disease.

42. The method of claim 1, wherein the apomorphine is administered in the absence of an anti-emetic.

43. The method of claim 1, wherein the apomorphine and the levodopa and/or dopamine agonist are administered sequentially, simultaneously, or concomitantly with each other.

44.-47. (canceled)

48. The method according to claim 1, wherein said administering comprises:
(A) administering apomorphine and the dopamine agonist by pulmonary inhalation using an inhalation device;
(B) administering apomorphine in combination with levodopa and/or dopamine agonist that is not apomorphine at a nominal dose by oral pulmonary inhalation using a dry powder passive or active inhaler;
(C) administering dopamine agonist and an additive material and/or carrier particles made from one or more excipient materials by oral pulmonary inhalation using an inhalation device; or
(D) administering a composition comprising apomorphine in combination with levodopa and/or dopamine agonist that is not apomorphine at a nominal dose by oral pulmonary inhalation using a dry powder passive or active inhaler, wherein the composition further comprises an additive material and/or carrier particles comprising one or more excipient materials.

49. A method of treating and/or preventing the symptoms of Parkinson's disease in a subject, the method comprising:
administering apomorphine to the subject by inhalation, wherein either (1) the maximum daily dose of apomorphine is less than 30 mg or (2) the apomorphine is delivered in a fine particle dose of 0.5 to 4.5 mg.

50. A method of reducing sleep loss, off-episodes, and/or dyskinesia in a subject with Parkinson's disease, the method comprising:
administering apomorphine by inhalation to reduce sleep loss, off-episodes, and/or dyskinesia in the subject.
